SEQUENCE LISTING (182 sequences)

SEQ ID NO: 1 - DNA, 789 bp, unidentified
  source 1..789: mol_type = genomic DNA; note = Caenibius tardaugens NBRC 16725
tcatgcctccggcacgccgggggcgccgggaaacccctgatagtatgtgcccgccataagcccgccatcgaccacgatttccgcgccgctcatgtacgaggcgtcatcgctggcgaggaacagtgaagccgccgcgacttcatctgtatcccccgcgcgctgcagcgggagatgcgcaaagctcgcatccagttcttcccgggtcacattgcccgcattgcccatcagcgtatcgatcgtgcccggatggaccgaattgacgcgaatgccgcgatggcccagttccatcgccgcaactttggtcaggccccgcacaccccatttgctggacacataggccccaaggctgttcccgccctgcaatcccgaaacggacgagatgttgacgatagaaccgctgccgcgggcgatcatgccgggcgccaccgccttgatcccgagaaactggctgacgagattgacgcggagcagacgttcataatcctccagcgtggtcgccagcaggctcctgaagatcagaacccccgcattgttgaccagcacgtccaccggcccgagatcggcttcggcctgcttcaccacctctgcccagcccgcttcgctggtcacgtcatggtgatagaagcacgccgcatcgcccagatcggcggccagtgcctgcccttcatccttgagcacatcggtgatggccaccttcgccccttcggcggcgaaaagacgggcagtcgccgcccccatgccgcgcgcaccgcccgtgataatcgcaactttgccagcaagccgacccat

SEQ ID NO: 2 - skipped

SEQ ID NO: 3 - protein, 6 aa, synthetic construct
LVNNAG

SEQ ID NO: 4 - skipped

SEQ ID NO: 5 - protein, 7 aa, synthetic construct
GGARGMG

SEQ ID NO: 6 - protein, 7 aa, synthetic construct
  VARIANT 3..4: note = X can be any amino acid
  VARIANT 6: note = X can be any amino acid
GGXXGXG

SEQ ID NO: 7 - protein, 7 aa, synthetic construct
  VARIANT 3..4: note = X can be any amino acid
  VARIANT 6: note = X can be any amino acid
GAXXGXG

SEQ ID NO: 8 - protein, 7 aa, synthetic construct
  VARIANT 2: note = X can be any amino acid
  VARIANT 4: note = X can be any amino acid
  VARIANT 6: note = X can be any amino acid
GXGXGXG

SEQ ID NO: 9 - protein, 7 aa, synthetic construct
  VARIANT 2: note = X can be any amino acid
  VARIANT 4: note = X can be any amino acid
  VARIANT 6: note = X can be any amino acid
GXAXGXG

SEQ ID NO: 10 - protein, 7 aa, synthetic construct
  VARIANT 2..3: note = X can be any amino acid
  VARIANT 6: note = X can be any amino acid
GXXRGXG

SEQ ID NO: 11 - protein, 7 aa, synthetic construct
  VARIANT 2..3: note = X can be any amino acid
  VARIANT 6: note = X can be any amino acid
GXXSGXG

SEQ ID NO: 12 - protein, 7 aa, synthetic construct
  VARIANT 2..4: note = X can be any amino acid
GXXXGMG

SEQ ID NO: 13 - protein, 7 aa, synthetic construct
  VARIANT 2..4: note = X can be any amino acid
GXXXGIG

SEQ ID NO: 14 - protein, 5 aa, synthetic construct
YVSSK

SEQ ID NO: 15-20 - skipped (6 entries)

SEQ ID NO: 21 - protein, 7 aa, synthetic construct
  VARIANT 4: note = X can be any amino acid
  VARIANT 6: note = X can be any amino acid
GGAXGXG

SEQ ID NO: 22 - protein, 7 aa, synthetic construct
  VARIANT 3: note = X can be any amino acid
  VARIANT 6: note = X can be any amino acid
GGXRGXG

SEQ ID NO: 23 - protein, 7 aa, synthetic construct
  VARIANT 3..4: note = X can be any amino acid
GGXXGMG

SEQ ID NO: 24 - protein, 7 aa, synthetic construct
  VARIANT 2: note = X can be any amino acid
  VARIANT 6: note = X can be any amino acid
GXARGXG

SEQ ID NO: 25 - protein, 7 aa, synthetic construct
  VARIANT 2: note = X can be any amino acid
  VARIANT 4: note = X can be any amino acid
GXAXGMG

SEQ ID NO: 26 - protein, 7 aa, synthetic construct
  VARIANT 2..3: note = X can be any amino acid
GXXRGMG

SEQ ID NO: 27 - protein, 7 aa, synthetic construct
  VARIANT 2: note = X can be any amino acid
GXARGMG

SEQ ID NO: 28 - protein, 7 aa, synthetic construct
  VARIANT 3: note = X can be any amino acid
GGXRGMG

SEQ ID NO: 29 - protein, 7 aa, synthetic construct
  VARIANT 4: note = X can be any amino acid
GGAXGMG

SEQ ID NO: 30 - protein, 7 aa, synthetic construct
  VARIANT 6: note = X can be any amino acid
GGARGXG

SEQ ID NO: 31 - protein, 7 aa, synthetic construct
GGGSGIG

SEQ ID NO: 32 - protein, 7 aa, synthetic construct
GGARGIG

SEQ ID NO: 33 - protein, 7 aa, synthetic construct
GGASGVG

SEQ ID NO: 34 - protein, 7 aa, synthetic construct
GAGSGMG

SEQ ID NO: 35 - protein, 7 aa, synthetic construct
GGSRGIG

SEQ ID NO: 36 - protein, 7 aa, synthetic construct
GGGQGLG

SEQ ID NO: 37 - protein, 7 aa, synthetic construct
GAGGGIG

SEQ ID NO: 38 - protein, 7 aa, synthetic construct
GAAGGIG

SEQ ID NO: 39 - protein, 7 aa, synthetic construct
GGARGMG

SEQ ID NO: 40-42 - skipped (3 entries)

SEQ ID NO: 43 - protein, 5 aa, synthetic construct
  VARIANT 2: note = X can be any amino acid
YXSSK

SEQ ID NO: 44 - protein, 5 aa, synthetic construct
  VARIANT 3: note = X can be any amino acid
YVXSK

SEQ ID NO: 45 - protein, 5 aa, synthetic construct
  VARIANT 4: note = X can be any amino acid
YVSXK

SEQ ID NO: 46 - protein, 5 aa, synthetic construct
YAASK

SEQ ID NO: 47 - protein, 5 aa, synthetic construct
YCASK

SEQ ID NO: 48 - protein, 5 aa, synthetic construct
YSASK

SEQ ID NO: 49 - protein, 5 aa, synthetic construct
YGASK

SEQ ID NO: 50 - protein, 5 aa, synthetic construct
YAMSK

SEQ ID NO: 51 - protein, 5 aa, synthetic construct
YSAAK

SEQ ID NO: 52 - protein, 5 aa, synthetic construct
YAAAK

SEQ ID NO: 53 - protein, 5 aa, synthetic construct
YSAAK

SEQ ID NO: 54 - protein, 5 aa, synthetic construct
YASSK

SEQ ID NO: 55 - DNA, 786 bp, Microbacterium resistens
  source 1..786: mol_type = genomic DNA; strain = MZT7
tcagaagtgcgtgccgccgtcgatgcggacctcggtgccggtgatgaacgcgccgtcggcgctcgccagcatcgccacgacgcccgcgaccgcctcggggccggcgaagccctgtccgatggccggggccagcttcaggaacaggctcatgtcggcgtcctcgggaagacccggtccggcgctctgtccgctgcgtccggagccgtcggtcatcccggacgagatggagccgggctgcacggcggtgaagcggatcccctgcttcgcgtactccgcggccagcgcgtgggtcatcgactgcaccccgcccttgctcgcggcgtacgcggccatgtagggatgggcgaacatcgccgacgtggagctgaagttcaccacggccgcgtcctgcccgtcgagcagggcggggatcgactcgcggatcatcaggaaggtgccgacgaggttcacgcgcaggatcgtctcgaactgctccagcgtcgtctccgccgtgtgcgaggagcgcaggatcccggcggcgttgaccagcacgtcgagcccgccgagggcgtccaccgcggtcgcgaccgcggccttcacggaggcctcgtcggcgacgttcacctccacggtggtcaggcgcgcggcggcgttgccggccttctcgacggtgtcggcgagacccgcggggctgacgtcggcggcgacgacgcgtcctccctcggcgagcatgcgcaggacggtggcctggccgatgccggatcctccgccggtgacgagcgcgcggcgaccttcgtagcggttcat

SEQ ID NO: 56 - DNA, 783 bp, Sphingomonas sp.
  source 1..783: mol_type = genomic DNA; strain = KC8
atgcggcgtctggaaggtaaggtagcgatcgtaaccggcggtgcgcgcggcattggcgaaggcatcgtccgtcgtttcgtgcaggaaggcgcgcgcgtcgtgatcgccgatctgctggaagccgaaggcacggcactggccgccgaactgggcgacgtgaccgtgttcgaaaagctcaacgtttcgtcgcgcgccgaatgggaccgcgtgatcgcggcgaccgaagcgcgcttcggcaagctcgattgcctggtcaacaatgccggcatcctgattttcaaggcgctcgacgatctgaccgaagaagaaatgcgccggatcatcgaggtcaacctgatcggcacgatgatcggcacgcaggccgtgatccccgcgatcgaacgcgccggtggcggcacgatcatcaacatgagttcggccgacggcatttcggcggcgaacggccttagcccctattgcgcgtcgaagttcggcgtgcgtggcttcagccagtcggtcgcgctggaacttggcccgcgcggcatccgcgtcaacacgatccacccgggcggcatctatacgccgctggccaaccaggccggggtgacccgcgaaatgttcgacatgggcttcaaggtctatccggcgcagcgcgcaggcgatccggcggatatcggcgcggcggccgcgttcctcgcgtcggacgatgccgcctattgcatcggcaccgaattgtcggtcgatggcggcctgaacgccggccattattacatggcggttccgggcgcgccgaagcagggctaa

SEQ ID NO: 57 - DNA, 765 bp, Comamonas testosteroni
  source 1..765: mol_type = genomic DNA; strain = ATCC 11996
ctatagccccatgcccagaatcgagttatctgcatgcagctcgctaccgctcatgacactggactcgtcgctggccaggaacaacaccagctgcgcgatacgctcgggcatataggcgcggccggcacggttgagcttggggtcatgcaagaccatttccttgctcacgccctttggcagcgaagcctgcatcatgggcgtatagatgccatcgggatgaatggaattgacgcgaatcgcatagccttgctttcgacagctcagtgcagcggcgcgggtcagtgccgacacggcggccttgctggcgctatagccggcgtattgctctatgggcagccagctcgataccgaggccatattgatgatggagcctcctgtctccttcatcgccgcaatgccctgctggcaaccaatgaagactgactcggtgttgatcttgagcaggcggctgaaatcctccagacgccctgtttccatgtcaccaggcagcaggatgccggcattgttgaccagcacattgagcgtgcccagacgccgctgcaccgccgccatcacaagcgtccagtccgcctcgctgctcacgtcatggcggacaaacatggagcgttcgcccagttcagccgccagttgctgccccgccgcttcattgatatcgctgaatgcgaccttggcgccttcgccaagaagcagcttcaccacctcaagacccacaccgctggcaccaccagtgaccagcgccaccttaccctgcaaacgatttgtcat

SEQ ID NO: 58 - DNA, 780 bp, Rhodococcus sp.
  source 1..780: mol_type = genomic DNA; strain = P14
tcagccgagctcccgggtgatgcggtggtagaggccgccggaggtcaggccgccgtcgatgacgatctcggtgcctgtgatgtacgtcgatctgtccgacaacaggaatgcgacggtttcggccacttcgtcggggtcggccgaccggccggcgggaacggatttcaggctctcgtcgacgaattccgtcgtacccgaggtcagcagcggcgtctcgacgagcccggggtggaccgagttgacgcgcacgtggtgttcggcgaactcgagtgcgccgaccttcgacaggccgcggacgccccatttgctggcgccgtaggcggcggcgaagtagccgagcataccggcgatcgaggagatgttgacgatcgagccaccaccgttgcacttgagcaacggcgcgcacgcccgcatgccgtagaacacgctgtcgaggttgatcgccatgacccgccgccagtcctcgacggaggtgtcctcgatcccgtatcgaaaactcaccccagcgttgttcaccagtccgtcgaggttcccgtcggcggcgtcgatcacctcggcgaatcgactccacgcgtcggggtcggtgacgtcgagccggtggaagtcggcggcaccgcccttgtcccggatctggtcgatgacggcttcgccggcggtggagtcgatgtcggcgacccacacatgcgccccgagggaggcgagcagctgtgcttcggcacggcccatcccgctgccggcaccggtgacggcgacggtcttgccggagaactcggtgttcat

SEQ ID NO: 59 - DNA, 750 bp, Pseudomonas putida
  source 1..750: mol_type = genomic DNA; strain = SJTE-1
atgtccaagcaacttacactcgaaggcaaagtggccctggttcagggcggttcccgaggcattggtgcagccatcgtgcggcgcctggcccgcgaaggcgcgcaagtggccttcacctatgtcagctctgccggcccggctgaagaactggttcgagaaattaccgaaaacggtggcaaagccttggccctgcgggctgacagcgctgatgccgcagccgtgcaactggcggtggatgacaccgtaaaagccttgggccggctggatatcctggtcaacaacgccggtgtgctggcagtggccccggtgacagagttcgacctggccgacttcgatcgcatgctggccgtgaacgtacgcagcgtgttcgtcgccagccaggccgccgcacgctatatgggccaggggggtcgtatcatcaacattggcagcaccaacgctgaacgcatgccgtttgccggtggcgcaccgtatgccatgagcaagtcggcactggttggcctgacccgcggcatggcacgcgacctcgggccgcagggcattaccgtgaacaatgtgcagccgggcccagtggacaccgacatgaacccggccagtggcgagtttgccgagagcctgattccgctgatggccattgggcggtatggcgagccggaggagattgccagctttgtggcatacctggccggccctgaagccgggtatatcaccggggccagcctgactgtagatggtgggtttgcagcctga

SEQ ID NO: 60 - DNA, 759 bp, Pseudomonas citronellolis
  source 1..759: mol_type = genomic DNA; strain = SJTE-3
ttacaatctcaacccgccatccagctccagcacgcggccggtgtagtagtcgttctcgaggatgtaggccaccgagtgggcgatctcgtccgggcggcccaggcgcttgagcgggatgccgctggtcatcttctccagggcctcgggcttcatgctggcgaccatgtcggtctcgatgaagcccggcgccacgccggccacgcggatgccgtagcgcgccagttccttggcccagaccacggtgtcggcggcgacgccggccttggccgcggagtagttggcctggcccatgttgccggcgcgggagatgctggagatgttgacgatcgcgccctggttgtccagctcgatcatcttcgccgccacttcgcgggtgcagaggaacacgccggtcaggttgacgtcgatcaccgcctgccactgggccaagctcatcttgctcagctggccgtccttgaccttgatggtcaggccgtcgcgcaggatgccggcgttgttcaccaggccgttgatggcgccgaagtcgctggccacctgggccaccatgtggctaacctgttcctcgttggccacgttgcacaggtaggcgcgggcgtcgccgccggcggccttgcaggcggcgacggcctcgtccagtttctcctggttcaggtcgaccagcgccagtttcgctcccttggcggcgaggaactcgcccatggcgcggcccaggccctggccgccgccggtgatgatgatgaccttgtcttgcaattgcat

SEQ ID NO: 61 - DNA, 897 bp, Sphingomonas sp.
  source 1..897: mol_type = genomic DNA; strain = C3-2
tcatcgcccatgctcctgcaccggccgtttcacaccgtcatgaaacggatagggctggcccagcatgccgagtgcaccgaaggcgccgcgcatctgcgcccggaattgggcctggatcgtctccagatcccagccttccgaacggatcagcgtcttgccgtaatgcggctgggaaagcagcgcgatgcggtctccccccgtgccgaacacctggccggacacgaaatcggcctcatccgatgccaggaaaacgatcatcggcccgttgagcgtcggatccggttgcaccgccaacacgccctcgcccggctttgtcgtcgccgacatgcgcgacatgcccagcgggctgaccgcgttcacccgaatgccataacgcgccagttccagcgcccaggtgtaggtcatcgatgcgatggcccctttcgccgccgcatagttggtctgcccgaaactgccgaaatgcgcggcagaggtggtgttgatcagcgcgccaccgcgcccctgttccaccatccgccgggcggcatgttgcgcgcaccagaaggtgcccttcacatgaacccgccaaaccgcgtcgaactcgtcgccggtcatcttcaggaacgtccggtccttcagattgcccgcattgttgacgaggatgtcgatcccgccaaacgcatcgaccgcggacttgacgagcgcggccgagctttcctcgcccgaaatatcatgtgtcgaggcgatcgcgcgcgcgccaaggctttcaatctccgaaaccacggcggccgccgcagcaccgtcgatgtcgttgaccacaacagccgcgcccgcgcgcgccaggtgcagcgcatggccacggccgatgccgccgcccgcgccggtaacgatcgccgattttccttccagaatcgtcat

SEQ ID NO: 62 - DNA, 900 bp, unidentified
  source 1..900: mol_type = genomic DNA; note = Caenibius tardaugens NBRC 16725
tcaaccctcctgcgtggaaggcggatagaccccgccgtggaaggggtagggtttgccgagaatgccgaacgcgccgaattgtccgggcatctgcgtctggaaatggcggcggatgctttccacatcccacccgcccggctggaccagagtcttgccgtaatgcggctgcaccatgtgcgacaggcgttcgccgcccgtgccgaatatctggcccgagacgttggcggcttcatcgctgcagagatagaccacggcgggtccgttccgtgacgggtcgaagtcctgaccgggcttgccgtcggcatcgcgataggttttcgacatggccgtggtgcccagcgggccgatggcattcacccgaatgccgtaacgggccagttccagcgcccaagtgtaggtcatcgatgcgatggcccctttggcggcggaataggcggtttgcccgaagctgccgaagtgcccaccagaggtcgtgttgatgatcgcgccgccggaaccctgcgcaaccatcgaccgggccgcagcctgcgtgcaccagaaggtccctttgacatggaccgtcaggatggcatcgagatcgtcatccgtcattttcagcaaggtcttgtcgcgcaggttgcccgcgttgttgacgagaatccgcacgcttccgaacgcggcccggcaggcttctaccagcgcttcggcgccttcgcgcgtgccgatgttgtgcgtcgataccaatgccgtgccacccgccgcggtcacttcgtcggccaatgcggcggcggcttcgccattgatgtcgttgatcaccacgggcgccccggcggcggcgagatgcagcacatgccctctgccgatgcccccagcagcgccggtgacaatggcgggacggccttcgagaagtgctgtgctcat

SEQ ID NO: 63 - DNA, 780 bp, Novosphingobium sp.
  source 1..780: mol_type = genomic DNA; strain = ES2-1
atgggaaagcttgcaggcaaggtggcgatcgtcaccggtggcgcgcggggcatgggcgcggcgacgagccggctgttcgtggccgagggcgcgaaagtggcgattgccgatgtgctggacgaggccggtgaagcactggccgcagagcttggcgatgcggcgcggttcttccatctcgacgtgaccgacgaggccaattgggccgcggttgtcagcgaagtggaagcaacgctgggaccggtcgacgcgctggtcaacaatgccggcatcctgatgttcaagagcctgctcgagacgaccaaggccgaatacgaaaaggtattgggcgtcaatctcgtcggcgagttcctgggcatcaaggcggtggcgccgggcatgatcgcgcgcgggcgcggttcgatcgtcaatgtctcttccgtggacgggatgaagggcgcgaacagtctggtcgcctatgcctcgtccaagtggggcgtgcgcgggctgaccaaggtggcggcgatggagctgggccacaagggcgttcgggtgaactcggtccaccccggcggggtggacacggtgatgtccaaccattccggcgcgccgcgcgaggaagtggacaagggctatggcaacgtgccgttgcagcgcatcggcgggccggaagaggtggccgcagccagcgccttccttgccagcgacgaggcgtcctacatccatggcgcagagatcgtggtcgacggcggaatgaccaccggcagctactacatgggcttccccggatcgccgggaatgtaa

SEQ ID NO: 64 - protein, 6 aa, synthetic construct
  VARIANT 1: note = X can be any amino acid
XVNNAG

SEQ ID NO: 65 - protein, 6 aa, synthetic construct
  VARIANT 2: note = X can be any amino acid
LXNNAG

SEQ ID NO: 66 - protein, 6 aa, synthetic construct
  VARIANT 3: note = X can be any amino acid
LVXNAG

SEQ ID NO: 67 - protein, 6 aa, synthetic construct
  VARIANT 4: note = X can be any amino acid
LVNXAG

SEQ ID NO: 68 - protein, 6 aa, synthetic construct
  VARIANT 5: note = X can be any amino acid
LVNNXG

SEQ ID NO: 69 - protein, 6 aa, synthetic construct
  VARIANT 6: note = X can be any amino acid
LVNNAX

SEQ ID NO: 70 - protein, 262 aa, unidentified
  source 1..262: note = Caenibius tardaugens NBRC 16725
MGRLAGKVAIITGGARGMGAATARLFAAEGAKVAITDVLKDEGQALAADLGDAACFYHHDVTSEAGWAEVVKQAEADLGPVDVLVNNAGVLIFRSLLATTLEDYERLLRVNLVSQFLGIKAVAPGMIARGSGSIVNISSVSGLQGGNSLGAYVSSKWGVRGLTKVAAMELGHRGIRVNSVHPGTIDTLMGNAGNVTREELDASFAHLPLQRAGDTDEVAAASLFLASDDASYMSGAEIVVDGGLMAGTYYQGFPGAPGVPEA

SEQ ID NO: 71 - protein, 261 aa, Microbacterium resistens
  source 1..261: note = strain MZT7
MNRYEGRRALVTGGGSGIGQATVLRMLAEGGRVVAADVSPAGLADTVEKAGNAAARLTTVEVNVADEASVKAAVATAVDALGGLDVLVNAAGILRSSHTAETTLEQFETILRVNLVGTFLMIRESIPALLDGQDAAVVNFSSTSAMFAHPYMAAYAASKGGVQSMTHALAAEYAKQGIRFTAVQPGSISSGMTDGSGRSGQSAGPGLPEDADMSLFLKLAPAIGQGFAGPEAVAGVVAMLASADGAFITGTEVRIDGGTHF

SEQ ID NO: 72 - protein, 260 aa, Sphingomonas sp.
  source 1..260: note = strain KC8
MRRLEGKVAIVTGGARGIGEGIVRRFVQEGARVVIADLLEAEGTALAAELGDVTVFEKLNVSSRAEWDRVIAATEARFGKLDCLVNNAGILIFKALDDLTEEEMRRIIEVNLIGTMIGTQAVIPAIERAGGGTIINMSSADGISAANGLSPYCASKFGVRGFSQSVALELGPRGIRVNTIHPGGIYTPLANQAGVTREMFDMGFKVYPAQRAGDPADIGAAAAFLASDDAAYCIGTELSVDGGLNAGHYYMAVPGAPKQG

SEQ ID NO: 73 - protein, 254 aa, Comamonas testosteroni
  source 1..254: note = strain ATCC 11996
MTNRLQGKVALVTGGASGVGLEVVKLLLGEGAKVAFSDINEAAGQQLAAELGERSMFVRHDVSSEADWTLVMAAVQRRLGTLNVLVNNAGILLPGDMETGRLEDFSRLLKINTESVFIGCQQGIAAMKETGGSIINMASVSSWLPIEQYAGYSASKAAVSALTRAAALSCRKQGYAIRVNSIHPDGIYTPMMQASLPKGVSKEMVLHDPKLNRAGRAYMPERIAQLVLFLASDESSVMSGSELHADNSILGMGL

SEQ ID NO: 74 - protein, 259 aa, Rhodococcus sp.
  source 1..259: note = strain P14
MNTEFSGKTVAVTGAGSGMGRAEAQLLASLGAHVWVADIDSTAGEAVIDQIRDKGGAADFHRLDVTDPDAWSRFAEVIDAADGNLDGLVNNAGVSFRYGIEDTSVEDWRRVMAINLDSVFYGMRACAPLLKCNGGGSIVNISSIAGMLGYFAAAYGASKWGVRGLSKVGALEFAEHHVRVNSVHPGLVETPLLTSGTTEFVDESLKSVPAGRSADPDEVAETVAFLLSDRSTYITGTEIVIDGGLTSGGLYHRITRELG

SEQ ID NO: 75 - protein, 249 aa, Pseudomonas putida
  source 1..249: note = strain SJTE-1
MSKQLTLEGKVALVQGGSRGIGAAIVRRLAREGAQVAFTYVSSAGPAEELVREITENGGKALALRADSADAAAVQLAVDDTVKALGRLDILVNNAGVLAVAPVTEFDLADFDRMLAVNVRSVFVASQAAARYMGQGGRIINIGSTNAERMPFAGGAPYAMSKSALVGLTRGMARDLGPQGITVNNVQPGPVDTDMNPASGEFAESLIPLMAIGRYGEPEEIASFVAYLAGPEAGYITGASLTVDGGFAA

SEQ ID NO: 76 - protein, 252 aa, Pseudomonas citronellolis
  source 1..252: note = strain SJTE-3
MQLQDKVIIITGGGQGLGRAMGEFLAAKGAKLALVDLNQEKLDEAVAACKAAGGDARAYLCNVANEEQVSHMVAQVASDFGAINGLVNNAGILRDGLTIKVKDGQLSKMSLAQWQAVIDVNLTGVFLCTREVAAKMIELDNQGAIVNISSISRAGNMGQANYSAAKAGVAADTVVWAKELARYGIRVAGVAPGFIETDMVASMKPEALEKMTSGIPLKRLGRPDEIAHSVAYILENDYYTGRVLELDGGLRL

SEQ ID NO: 77 - protein, 298 aa, Sphingomonas sp.
  source 1..298: note = strain C3-2
MTILEGKSAIVTGAGGGIGRGHALHLARAGAAVVVNDIDGAAAAAVVSEIESLGARAIASTHDISGEESSAALVKSAVDAFGGIDILVNNAGNLKDRTFLKMTGDEFDAVWRVHVKGTFWCAQHAARRMVEQGRGGALINTTSAAHFGSFGQTNYAAAKGAIASMTYTWALELARYGIRVNAVSPLGMSRMSATTKPGEGVLAVQPDPTLNGPMIVFLASDEADFVSGQVFGTGGDRIALLSQPHYGKTLIRSEGWDLETIQAQFRAQMRGAFGALGMLGQPYPFHDGVKRPVQEHGR

SEQ ID NO: 78 - protein, 299 aa, unidentified
  source 1..299: note = Caenibius tardaugens NBRC 16725
MSTALLEGRPAIVTGAAGGIGRGHVLHLAAAGAPVVINDINGEAAAALADEVTAAGGTALVSTHNIGTREGAEALVEACRAAFGSVRILVNNAGNLRDKTLLKMTDDDLDAILTVHVKGTFWCTQAAARSMVAQGSGGAIINTTSGGHFGSFGQTAYSAAKGAIASMTYTWALELARYGIRVNAIGPLGTTAMSKTYRDADGKPGQDFDPSRNGPAVVYLCSDEAANVSGQIFGTGGERLSHMVQPHYGKTLVQPGGWDVESIRRHFQTQMPGQFGAFGILGKPYPFHGGVYPPSTQEG

SEQ ID NO: 79 - protein, 259 aa, Novosphingobium sp.
  source 1..259: note = strain ES2-1
MGKLAGKVAIVTGGARGMGAATSRLFVAEGAKVAIADVLDEAGEALAAELGDAARFFHLDVTDEANWAAVVSEVEATLGPVDALVNNAGILMFKSLLETTKAEYEKVLGVNLVGEFLGIKAVAPGMIARGRGSIVNVSSVDGMKGANSLVAYASSKWGVRGLTKVAAMELGHKGVRVNSVHPGGVDTVMSNHSGAPREEVDKGYGNVPLQRIGGPEEVAAASAFLASDEASYIHGAEIVVDGGMTTGSYYMGFPGSPGM

SEQ ID NO: 80 - protein, 262 aa, unidentified
  source 1..262: note = Caenibius tardaugens, Caenibius tardaugens NBRC 16725
MGRLSGKVAIVTGGARGMGAATVRLFVAEGARVAITDVLDAEGNALAAELGDAARFYHQDVTSEAGWAEVVKQTEADLGPVDVLVNNAGILMFKSLLATSLEEYERVLRVNLVGEFLGIKAVAPGMIARGKGSIVNVSSVDGMKGANSLGAYASSKWGVRGLTKVAAMELGHRGIRVNSVHPGGVDTAMTNHNNASRETVNERFTNVPLQRVGGPEEVAAASLFLASDDASYMTGAEIVVDGGMTIGVYYEGFPGAPGVPEA

SEQ ID NO: 81 - protein, 262 aa, Novosphingobium sp.
MGRLSGKVAIITGGARGMGAATSRLFAAEGAKVAITDVLEAEGTALAAELGDAARFYRHDVTDEAAWAEIVKQVEADLGPVDVLVNNAGILMFRSLLSTTLEDYERVLRVNLIGEFLGIKAVAPGMIARGKGSIVNISSVDGMKGANGLAAYASSKWGVRGLTKVAAMELGHRGIRVNSVHPGGVDTVMTNQNNSAREEVNQRFGNVPLQRVGGPEEVAAASLFLASDDSSYLAGAEIVVDGGMIVGQYYEGFPGAPGVPEA

SEQ ID NO: 82 - protein, 262 aa, unidentified
  source 1..262: note = Caenibius sp. WL
MGRLSGKVAIVTGGARGMGAATSRLFAAEGAKVAIADVLDAEGAALAAELGDAARFYHHDVTNEAGWADLVAGVESDLGPVDVLVNNAGILMFRTLIGTTLDEYERVLKVNLVGQFLGIKAVAPGMIARGRGSIVNISSVSGMQAGNCLGAYASSKWGVRGLTKVAALELGHRGIRVNSIHPGTIDTAMGNQGAMSREELDATFAHLPLQRAGGQEEVAAASLFLASDDASYMAGAEIVVDGGLMAGQFYQGMPGAPGVAES

SEQ ID NO: 83 - protein, 262 aa, unidentified
  source 1..262: note = Caenibius sp. WL
MGRLSGKVAIVTGGARGMGAATSRLFAAEGAKVAITDVLEEDGKALAAELGDAARFYRHDVTSESEWAAIVQQVEADLGPVDVLVNNAGILMFKSLLATTLEDYERVLKVNLVGEFLGIKAVAPGMIARGKGSIVNISSVDGMKGANGLVAYASSKWGVRGLTKVAALELGHRGVRVNSVHPGGVDTAMTNHDNTARDKVNERFTNIPLQRVAGPEEVAAASLFLASDDASYLAGAEIVVDGGMTSGQYYPGFPGAPGVPEA

SEQ ID NO: 84 - protein, 262 aa, unidentified
  source 1..262: note = Croceicoccus estronivorus
MGRLSGKVAIITGGARGMGAATSRLFVAEGAKVAITDVLEEEGNALAAELGDAARFYRHDVTSEAEWAGIVKQVEADLGPVDVLVNNAGILMFKSLLSTTLEDYERVLRVNLIGEFLGIKAVAPGMIERGKGSIVNISSVDGMKGANGLCAYASSKWGVRGLTKVATMELGHRGIRVNSVHPGGVDTAMTNFDSSSRDKVNERFTNVPLQRVSGPEEVAAASLFLASDESSYLAGAEIVVDGGMTVGQYYEGFPGAPGVPEA

SEQ ID NO: 85 - protein, 259 aa, unidentified
  source 1..259: note = Comamonas fluminis
MNRLAGKVAIITGGARGMGAATCRLFVAQGAKVAIADLLNSEGQALAAELGDAARFYHHDVTSEESWAALVQGVQRDLGAVDVLVNNAGMLMFRTLLETTLQEYERVLKVNLVGEFLGIKAVAPGMVERGRGSIINISSVDGMKGANGLAAYASSKWGVRGLTKVAAMELGHRGVRVNSVHPGGVDTVMGNPGQRSRNEVNQGFTNIALQRVGEPHEVAAATLFLASDESSYMVGAEIVVDGGMTAGHYYQGLPGAPGV

SEQ ID NO: 86 - protein, 259 aa, Comamonas sp.
MNRLAGKVAIITGGARGMGAATCRLFVEQGAKVAIADLLEAEGRALAAELGDAARFYHHDVSSEESWAALMRHVQQDLGAVDVLVNNAGMLMFRTLLETTLQEYERVLKVNLVGEFLGIKAVTPGMIERGKGSIVNISSVDGMKGANGLAAYASSKWGVRGLTKVAAMELGHRGVRVNSVHPGGVDTVMGNPGQRTRTEVNQGFTNIPLQRVGEAHEVAAATLFLASDESSYMVGAEIVVDGGMTTGHYYQGLPGAPGV

SEQ ID NO: 87 - protein, 259 aa, unidentified
  source 1..259: note = Comamonas testosteroni, Comamonas testosteroni ATCC 11996, Comamonas sp. AG1104
MNRLAGKVAIITGGARGMGAATCRLFVQEGAKVAVADMLDVEGQALAAELGDAARFYHHDVTSEESWAGLMEQAQQDLGAIDVLVNNAGVLMFRTLLDTSLADYERVLKVNLVGEFLGIKAVAPGMIERGRGSIINLSSVDGMKGANGLAAYASSKWGVRGLTKVAAMELGHRGVRVNSVHPGGVDTAMGNPGQRAREEVNQGFANIALQRVGDPAEVAAATLFLASDESSYMAGAEIVVDGGMTAGHYYAGLPGAPGM

SEQ ID NO: 88 - protein, 259 aa, unidentified
  source 1..259: note = Sphingobium agri
MGRLQGKVAIITGGARGMGAATSRLFAEEGAKVAITDVLEEDGAQLAAELGDAARFYRHDVTSEEGWADLVRTVEADLGPVDILVNNAGILLFRTLLDTTLADYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVNERFGNVPLQRVGAPEEVARATLFLASDEASYLAGAEIAVDGGMLTGQYYVQFPGAPGV

SEQ ID NO: 89 - protein, 259 aa, unidentified; identical to SEQ ID NO: 88
  source 1..259: note = Sphingobium

SEQ ID NO: 90 - protein, 259 aa, Sphingobium sp.
  source 1..259: note = strain TomTYG65
MGRLEAKVAIITGGARGMGAATSRLFVQEGAKVAIADVLDAEGTALASELGDAARFYHHDVTSEEGWAELVRTVEADFGPIDILVNNAGILLFRTLLDTSLADYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVNERFGNVPLQRVGAPEEVARATLFLASDDASYLAGAEIAVDGGMLTGQYYENFPGAPGV

SEQ ID NO: 91 - protein, 259 aa, Sphingobium sp.; identical to SEQ ID NO: 88
  source 1..259: note = strain TomMM35A

SEQ ID NO: 92 - protein, 259 aa, Novosphingobium sp.
  source 1..259: note = strain Chol11
MGRLQGKVAIITGGARGMGAATSRLFVEEGAKVAITDVLEEDGAQLAAELGDAARFYRHDVTSEEGWADLVRTVEADLGPVDILVNNAGILLFRTLLDTTLADYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVNERFGNVPLQRVGAPEEVARATLFLASDEASYLAGAEIAVDGGMLTGQYYVQFPGAPGV

SEQ ID NO: 93 - protein, 259 aa, unidentified
  source 1..259: note = Sphingobium herbicidovorans, Sphingobium herbicidovorans NBRC 16415
MGRVQGKVAIITGGARGMGAATCRLFVQEGARVAIADVLDEDGAALASELGDAARFYHHDVTSEEGWADLVRAVEADLGPVDILVNNAGILLFRTLLDTSLADYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVNERFGNVPLQRVGAPEEVARATLFLASDDASYLAGAEIAVDGGMLTGQYYEQFPGAPGV

SEQ ID NO: 94 - protein, 259 aa, unidentified
  source 1..259: note = Novosphingobium aromaticivorans, Novosphingobium aromaticivorans DSM 12444
MGRLSGKVAIVTGGARGMGAATSRLFVAEGAKVAIADVLDEAGEALAAELGDAARFFKLDVTSEDNWASVVSEVEAALGPVDVLVNNAGILMFKSLLETTKADYEKVLGVNLVGEFLGIKAVAPGMIARGKGSIVNISSVDGMKGANSLVAYASSKWGVRGLTKVAAMELGHKGIRVNSVHPGGVDTVMSNHSGAAREDVDKGYANVPLQRIGGPEEVAAASLFLASDDASYLHGAEIVVDGGMTVGTYYMGFPGSPGM

SEQ ID NO: 95 - protein, 259 aa, Sphingobium sp.
  source 1..259: note = strain TomTYG46
MGRLQGKVAIITGGARGMGAATSRLFAEEGAKVAIADVLDDVGTQLATELGDVACFYHHDVTSEQGWAELVGAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTAMTNHDGSTREKVSERFGNVPLQRVGAPEEVARATLFLASDEASYLAGAEIAVDGGMLTGQYYAQFPGAPGV

SEQ ID NO: 96 - protein, 259 aa, unidentified
  source 1..259: note = unclassified Sphingobium, Sphingomonadales bacterium 56, Sphingomonadales bacterium 58, Sphingobium sp. S8, Sphingobium sp. S6
MRRLQGKVAIITGGARGMGAATSRLFAEEGAKVAITDVLEEDGAQLAAELGDAARFYRHDVTSEEGWADLVRTVEADLGPVDILVNNAGILLFRTLLDTTLADYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVNERFGNVPLQRVGAPEEVARATLFLASDEASYLAGAEIAVDGGMLTGQYYVQFPGAPGV

SEQ ID NO: 97 - protein, 259 aa, Sphingobium sp.
  source 1..259: note = strain TomTYG73
MGRLQGKVAIITGGARGMGAATSRLFAEEGAKVAITDVLDEVGTQLATELGDAARFYHHDVTSEQGWAELVDAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVSERFGNVPLQRVGAPEEVARATLFLASDEASYLAGAEIAVDGGMLTGQYYAQFPGAPGV

SEQ ID NO: 98 - protein, 259 aa, Sphingobium sp.
  source 1..259: note = strain EM0848
MNRLHNKVAIITGGARGMGAATSRLFVAEGAKVAIADLLDEAGEALAAELGDAARFYHHDVTSEEGWANLVAAVEADLGPVDILVNNAGILLFRTLLDTTLEDYEKVLNVNLVGEFLGIKAVAPGMIARGNGSIVNISSVDGMKGANGLAAYSSSKWGVRGLTRVAALELGHRGVRVNSVHPGGVDTVMTNHEGRSRDSVSERFRNIPLQRVGGPEEVAKATLFLASDDASYLAGAEIAVDGGMLTGHYYEGLPGAPGV

SEQ ID NO: 99 - protein, 259 aa, Sphingobium sp.
  source 1..259: note = strain RC1
MGRLQGKVAIITGGARGMGAATSRLFAEEGAKVAITDVLDEVGTQLATELGDAARFYHHDVTSEQGWAELVGAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTAMTNHDGSTREKVSERFGNVPLQRVGAPEEVARATLFLASDEASYLAGAEIAVDGGMLTGQYYAQFPGAPGV

SEQ ID NO: 100 - protein, 259 aa, unidentified
  source 1..259: note = Sphingobium sp. TomMM15, Sphingobium sp. TomTYG75
MGRLQGKVAIITGGARGMGAATSRLFAEEGAKVAITDVLDEVGTQLATELGEAARFYHHDVTSEQGWAELVGAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVSERFGNVPLQRVGAPEEVARATLFLASDEASYLAGAEIAVDGGMLTGQYYAQFPGAPGV

SEQ ID NO: 101 - protein, 259 aa, Sphingobium sp.
  source 1..259: note = strain TomTYG74
MGRLQGKVAIITGGARGMGAATSRLFVEEGAKVAITDVLDEVGTQLATELGDAARFYHHDVTSEQGWAELVGAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVSERFGNVPLQRVGAPEEVARATLFLASDEASYLAGAEIAVDGGMLTGQYYAQFPGAPGV

SEQ ID NO: 102 - protein, 259 aa, Sphingobium sp.
  source 1..259: note = strain TomTYG72
MGRLQGKVAIITGGARGMGAATSRLFAEEGAKVAITDVLDEVGTQLATELGDAARFYHHDVTSEQGWAELVGAVETDLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVSERFGNVPLQRVGAPEEVARATLFLASDEASYLAGAEIAVDGGMLTGQYYPQFPGAPGV

SEQ ID NO: 103 - protein, 259 aa, unidentified
  source 1..259: note = Sphingobium sp. Cam5-1, Sphingobium sp. TomTYG64, Sphingobium sp. TomTYG45
MGRLQGKVAIITGGARGMGAATSRLFVEEGAKVAITDVLDEVGTQLAAELGEAARFYHHDVTSEQGWAELVGAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVSERFGNVPLQRVGAPEEVARATLFLASDEASYLAGAEIAVDGGMLTGQYYAQFPGAPGV

SEQ ID NO: 104 - protein, 259 aa, Sphingobium sp.
MGRLQDKVAIITGGARGMGAATSRLFAQEGAKVAIADVLEEDGAQLAAELGDSARYYRHDVTSEEGWADLVRSVEADLGPVDILVNNAGILLFRTLLDTTLADYERVLKVNLTGEFLGIKAVAPGMIARGKGAIVNISSVDGMKGANGLAAYSSSKWGVRGLTRVAALELGHRGIRVNSVHPGGVDTVMTNQDGSTREKVNERFGNVPLQRVGAPEEVARATLFLASDEASYLAGAEIAVDGGMLTGQYYVQFPGAPGV

SEQ ID NO: 105 - protein, 259 aa, Sphingobium sp.
  source 1..259: note = strain HWE2-09
MGRLEGKVAIVTGGARGMGAATCRLFVAEGAKVAIADLLDDAGATLAAELGDAARFYHHDVTSEAAWASLVGAVEADLGPIDILVNNAGILLFRTLLETTLADYEKVLKVNLVGEFLGIKAVAPAMVARGRGSIVNISSVDGMKGANGLAAYSSSKWGVRGLTRVAALELGHKGVRVNSVHPGGVDTVMTNYDGRSRDAVSERFGNIPLQRVGAPEEVAKATLFLASDDASYLAGAEIAVDGGMLTGQYYEGFPGAPGV

SEQ ID NO: 106 - protein, 259 aa, Novosphingobium sp.
  source 1..259: note = strain THN1
MGKLAGKVAIVTGGARGMGAATSRLFVAEGAKVAIADVLDEAGEALAAELGDAARFFHLDVTDEANWAAVVSEVEATLGPVDALVNNAGILMFKSLLETSKADYERVLGVNLVGEFLGIKAVAPGMIARGRGSIVNISSVDGMKGANSLVAYASSKWGVRGLTKVAAMELGHKGVRVNSVHPGGVDTVMSNHTGAAREEVDKGYANVPLQRIGGPEEVASASAFLASDEASYIHGAEIVVDGGMTVGSYYMGFPGSPGM

SEQ ID NO: 107 - protein, 259 aa, unidentified
  source 1..259: note = Sphingobium estronivorans
MARLKDKVAIVTGGARGMGAATSRLFAAEGAKVAIADLLDEAGEALAAELGDTARFYHHDVTSEEGWASLVAAVEADFGPVDILVNNAGILLFRSLLETTREEYEKVLNVNLVGEFLGIKAVAPGMVARGKGSIVNISSVDGMKGANGLAAYASSKWGVRGLTRVAALELGHRGVRVNSVHPGGVDTVMTNYEGRSREAVSERFRNIPLQRVGGPEEVAKATLFLASDDASYLAGAEIAVDGGMLTGQYYEGMPGAPGV

SEQ ID NO: 108 - protein, 259 aa, unidentified
  source 1..259: note = Sphingobium, Sphingobium sp. EP60837, Sphingobium tyrosinilyticum
MGRLQGKIAIITGGARGMGAATSRLFIEEGAKVAIADVLDADGAELASELGDAARFYHHDVTSEDGWTELVRAVEADLGPTDILVNNAGILLFRTLLDTSLGDYERVLKVNLTGAFLGIKAVAPGMIARGKGAIVNISSVDGMKGANGLAAYSSSKWGVRGLTRVAALELGHRGIRVNSVHPGGVDTVMTNQDGSTREKVNERFGNVPLQRVGAPEEVARATLFLASDEASYLAGAEIAVDGGMLTGQYYENFPGAPGV

SEQ ID NO: 109 - protein, 259 aa, unidentified
  source 1..259: note = Sphingobium sp. TomMM7A, Sphingobium sp. TomTYG42, Sphingobium sp. TomTYG43, Sphingobium sp. TomTYG71
MGRLRGKVAIITGGARGMGAATSRLFVEEGAKVAITDVLDEVGTQLATELGKAARFYHHDVTSEQGWAELVGAVEADLGPVDILVNNAGILLFRTLLDTSLAEYERVLKVNLTGEFLGIKAVAPGMIARGKGAIVNISSVDGMKGANGLAAYSSSKWGVRGLTRVAALELGHRGIRVNSVHPGGVDTVMTNHDGSTREKVSERFGNVPLQRVGAPEEVARATLFLASDEASYLAGAEIAVDGGMLTGQYYAQFPGAPGV

SEQ ID NO: 110 - protein, 258 aa, unidentified
  source 1..258: note = Sphingomonadales bacterium
GKLAGKVAIVTGGARGMGAATSRLFAAEGAKVAIVDMLEAEGIALAAELGETTRFWKHDVTSEAGWTALVAEVEAAFGGVDVLVNNAGIIIFKGLLDTSAEEYRRVLDVNLVGEFLGIKAVAPGMIARGKGSIINVSSVDGMKGANCLVAYSSSKWGVRGLTKVAAMELGHRGIRVNSVHPGGVDTIMSNHANVAREELNASYTNVPLQRIGGPEEVAAATLFLASDDASYIHGTEIVVDGGMTTGVYYTGFPGAPGV

SEQ ID NO: 111 - protein, 259 aa, Novosphingobium sp.; identical to SEQ ID NO: 79
  source 1..259: note = strain ES2-1

SEQ ID NO: 112 - protein, 259 aa, Novosphingobium sp.
  source 1..259: note = strain ERW19
MGKLAGKVALVTGGARGMGAATSRLFVAEGAKVAIADVLDEAGEALAAELGDAARFFHLDVTDEANWAAVVSDVEATLGPVDALVNNAGILMFKSLLETSKADYEKVLGVNLVGEFLGIKAVAPGMIARGRGSIVNISSVDGMKGANSLVAYASSKWGVRGLTKVAAMELGHKGVRVNSVHPGGVDTVMSNHSGAAREEVDKGYANVPLQRIGGPEEVASASAFLASDEASYIHGAEIVVDGGMTVGSYYMGFPGSPGM

SEQ ID NO: 113 - protein, 259 aa, Novosphingobium subterraneum
MGKLAGKVAIVTGGARGMGAATSRLFVAEGAKVAIADVLDEAGEALAAELGDAARFFHLDVTDEANWAAVVGEVEATLGPVDALVNNAGILLFKSLFETTKADYEKVLGVNLVGEFLGIKAVAPGMIARGRGSIVNVSSVDGMKGANSLVAYASSKWGVRGLTKVAAMELGHKGVRVNSVHPGGVDTVMSNHSGAPREEVDKGYGNVPLQRIGGPEEVAAASAFLASDEASYIHGAEIVVDGGMTTGSYYMGFPGSPGM

SEQ ID NO: 114 - protein, 258 aa, unidentified
  source 1..258: note = Pseudomonadota bacterium
MGKLDGKVAIVTGGARGMGEATTRRFVAEGAKVAIADVLEEQGQALAAELGDAARFWKLDVTSEDDWARVVTEVEAELGPVDALVNNAGILMFKGLLETTKADYERVLGVNLVGEFLGIKAVAPGMIARGRGAIVNVSSVDGMKGANSLVAYASSKWGVRGLTKVAAMELGHKGIRVNSVHPGGVDTVMSNHNAAPREEVDKGYANVPLQRIGGPEEVAAASAFLASDDASYIHGAEIVVDGGMTVGTYYQGFPGSPG

SEQ ID NO: 115 - protein, 258 aa, unidentified
  source 1..258: note = Sphingomonadales bacterium
MGKLDGKVAIVTGGARGMGEATTRRFVAEGAKVAIADVLEEQGQALAAELGDAARFWKLDVTSEDDWARVVAEVEAELGPVDALVNNAGILMFKGLLETTKADYERVLGVNLVGEFLGIKAVAPGMIARGRGAIVNVSSVDGMKGANSLAAYASSKWGVRGLTKVAAMELGHKGIRVNSVHPGGVDTVMSNHNAAPREEVDKGYANVPLQRIGGPEEVAAASAFLASDDASYIHGAEIVVDGGMTVGSYYQGFPGSPG

SEQ ID NO: 116 - protein, 259 aa, Comamonas sp.
MNRLAGKTAIITGGARGMGAATCRLFVEQGAKVAIADMLEAEGRALAAELGDAARFYPHDVTSEESWAALVRNVEKDLGVVDVLVNNAGVLLFKTLLETSLADYERVLKVNLVGEFLGIKAVAPGMIERGRGSIINLSSVEGMKGANGLAAYASSKWGVRGLTKVAAMELGHRGVRVNSVHPGGVDTAMGNPGQRSREQVNQGFTNIALQRVGEPSEVAAATLFLASDESSYMVGAEIVVDGGMTAGHYYVGMPGAPGM

SEQ ID NO: 117 - protein, 259 aa, Comamonas sp.
MNRLAGKTAIITGGARGMGAATCRLFVEQGAKVAIADMLEAEGRALAAELGDAARFYPHDVTSEESWAALVRNVEKDLGVVDVLVNNAGVLLFKTLLETSLADYERVLKVNLVGEFLGIKAVAPGMIERGRGSIINLSSVDGMKGANGLAAYASSKWGVRGLTKVAAMELGHRGVRVNSVHPGGVDTAMGNPGQRSREQVNQGFTNIALQRVGEPSEVAAATLFLASDESSYMVGAEIVVDGGMTAGHYYVGMPGAPGM

SEQ ID NO: 118 - protein, 259 aa, Comamonas sp.
  source 1..259: note = strain 26
MNRLAGKTAIITGGARGMGAATCRLFVEQGAKVAIADMLEAEGRALAAELGDAARFYPHDVTSEESWAALVRNVEKDLGVVDVLVNNAGVLLFKTLLDTSLADYERVLKVNLVGEFLGIKAVAPGMIERGRGSIVNLSSVDGMKGANGLAAYASSKWGVRGLTKVAAMELGHRGVRVNSVHPGGVDTAMGNPGQRSREQVNQGFTNIALQRVGEPSEVAAATLFLASDESSYMVGAEIVVDGGMTAGHYYVGMPGAPGM

SEQ ID NO: 119 - protein, 259 aa, unidentified
  source 1..259: note = Sphingobium quisquiliarum P25, Sphingobium quisquiliarum
MNRLQGKTAIITGGARGMGEATSRLFAAEGAKVAIADLLDDQGAQLAAELGGAARFYHHDVTSEADWASLTAAVEADFGPADILVNNAGILLFRTLFQTTLDDYLKVLKVNLAGAFLGIKAVGPGMAARGKGSIVNISSVDGMKGANGLGAYASSKWGLRGLTKVAAMELGHKGVRVNSVHPGGVDTMMGNLDSRSRDEVNKDYGNVPLQRIGGPEEIAKASLFLASDDASYLTGAEIVVDGGMIVGHYYEGFPGAPGV

SEQ ID NO: 120 - protein, 259 aa, unidentified
  source 1..259: note = Sphingomonadales bacterium
MGRLEGKVAIVTGGARGMGAATVRRFVAEGARVAIADVLDEAGKALAAELGPATGFWKLDVTSEENWARVVAEVEAALGPVDVLVNNAGILMFKSLLETTKADYERVLGVNLVGEFLGIKAVAPGMIARGRGAIVNVSSVDGMKGANSLGAYASSKWGVRGLTKVAAMELGHKGVRVNSVHPGGVDTVMSNHAAAPRDEVDKAYGHVPLQRIGGPEEVAAASAFLASDDASYIHGAEIVVDGGMTVGSYYAGFPGAPGV

SEQ ID NO: 121 - protein, 259 aa, unidentified
  source 1..259: note = unclassified Novosphingobium, Novosphingobium sp., Novosphingobium sp. 12-62-10, Novosphingobium sp. 28-62-57, Novosphingobium sp. 17-62-9
MGKLTGKVAIITGGARGMGAATSRLFVAEGAKVAIADVLEEDGAALAAELGDAARFYKLDVTNEDDWARVVAAAEADLGPVDALVNNAGILMFKSILETTKADYERVLGVNLVGEFLGIKAVAPGMIARGKGSIVNISSVDGMKGANSLVAYASSKWGVRGLTKVAAMELGHKGIRVNSVHPGGVDTVMSNHTGAPRAEIDKQYSNVPMQRIGGPEEVAAASAFLASDESSYCHGTEIVVDGGMTVGTYYMGFPGSPGM

SEQ ID NO: 122 - protein, 259 aa, Novosphingobium taihuense
MGKLEGKVAIVTGGGRGMGAATVRRFVADGAKVAIADVLEAEGSALAAELGDAARFYRLDVTNEENWAKVVSAVEADLGPVSVLVNNAGILMFKSLLETTKADYERVLGVNLVGEFLGIKAVAPGMIARGKGSIVNISSVDGMKGANSLVAYASSKWGVRGLTKVAAMELGHKGVRVNSVHPGGVDTVMSNHNAAPRAEVDKGYANVPLQRIGGPEEVAAASAFLASDDASYMHGAELVVDGGMTVGTYYMGFPGSPGM

SEQ ID NO: 123 - protein, 259 aa, Comamonas sp.
MNRLAGKTAIITGGARGMGAATCRLFVEQGAKVAIADMLEAEGRALAAELGDAARFYPHDVTSEESWAALVRNVEKDLGAADVLVNNAGVLLFKTLLETSLADYERVLKVNLVGEFLGIKAVAPGMIERGRGSIINLSSVDGMKGANGLAAYASSKWGVRGLTKVAAMELGHRGVRVNSVHPGGVDTAMGNPGQRSREQVNQGFTNIALQRVGEPSEVAAATLFLASDESSYMVGAEIVVDGGMTAGHYYVGMPGAPGM

SEQ ID NO: 124 - protein, 259 aa, Novosphingobium sp.
  source 1..259: note = strain CCH12-A3
MGKLAGKVAIVTGGARGMGAATSRLFVAEGAKVAIADVLDEAGEALAAELGYAARFFHLDVTDEANWAAVVGEVEATLGPVDALVNNAGILLFKSLLETTKADYEKVLGVNLVGEFLGIKAVAPGMIARGRGSIVNVSSVDGMKGANSLVAYASSKWGVRGLTKVAAMELGHKGVRVNSVHPGGVDTVMSNHSGAPREEVDKGYGNVPLQRIGGPEEVAAASAFLASDEASYIHGAEIVVDGGMTTGSYYMGFPGSPGM

SEQ ID NO: 125 - protein, 259 aa, Comamonas sp.
MNRLAGKTAIITGGARGMGAATCRLFVEQGAKVAIADMLEAEGRALAAELGDAARFYPHDVTSEESWAALVRNVEKDLGVADVLVNNAGVLLFKTLLETSLADYERVLKVNLVGEFLGIKAVAPGMIERGRGSIINLSSVDGMKGANGLAAYASSKWGVRGLTKVAAMELGHRGVRVNSVHPGGVDTAMGNPGQRSREQVNQGFTNIALQRVGEPSEVAAATLFLASDESSYMVGAEIVVDGGMTAGHYYVGMPGAPGM

SEQ ID NO: 126 - protein, 259 aa, Sphingobium sp.
  source 1..259: note = strain DC-2
MNRLQGKTAIITGGARGMGEATSRLFAAEGAKVAIADLLDDQGAQLAAELGGAARFYHHDVTSEADWASLTAAVEADFGPADILVNNAGILLFRTLFQTSLDDYLKVLKVNLAGAFLGIKAVGPGMAARGKGSIVNISSVDGMKGANGLGAYASSKWGLRGLTKVAAMELGHKGVRVNSVHPGGVDTMMGNLDSRSRDEVNKDYGNVPLQRIGGPEEIAKASLFLASDDASYLTGAEIVVDGGMIVGHYYEGFPGAPGV

SEQ ID NO: 127 - protein, 259 aa, unidentified
  source 1..259: note = Novosphingobium sp., Novosphingobium sp. 35-62-5, Novosphingobium sp. 16-62-11
MGKLAGKVAIITGGARGMGAATSRLFVAEGAKVAIADVLEEDGAVLAAELGDAARFYKLDVTNEDDWARVVTAAEADLGPIDALINNAGILMFKSILETTKADYERVLGVNLVGEFLGIKAVAPGMIARGKGSIVNISSVDGMKGANSLVAYASSKWGVRGLTKVAAMELGHKGVRVNSVHPGGVDTVMSNHSAAPRAEIDKQYSNVPMQRIGGPEEVAAASAFLASDESSYCHGTEIVVDGGMTVGTYYMGFPGSPGM

SEQ ID NO: 128 - protein, 259 aa, unidentified
  source 1..259: note = Comamonas suwonensis
MNRLAGKTAIITGGARGMGAATCRLFVEQGAKVAIADMLEVEGRALAAELGNAARFYPHDVTSEESWAALMRNVEKDLGAVDVLVNNAGVLLFKTLLDTSLADYERILKVNLVGEFLGIKAVAPGMVERGRGSIINLSSVDGMKGANGLAAYASSKWGVRGLTKVAAMELGHRGVRVNSVHPGGVDTAMGNPGQRSREQVNQGFTNIALQRVGESSEVAAATLFLASDESSYMVGAEIVVDGGMTAGHYYVGMPGAPGM

SEQ ID NO: 129 - protein, 258 aa, unidentified
  source 1..258: note = Comamonas sp., Comamonas sp. NoAH
MGRLSGKVALVTGGARGMGAATSRLFVAEGAKVAIADVLDAPGQALAEELGSAARFYTLDVTSEANWASVVEQVEADFGPIDVLVNNAGILLFRTLLETSLQDYERVLRVNLTGEFLGIKSVGAGMVARRKGSIVNISSIDGMKGANGLAAYASSKWGVRGLTKVAAMELGHHGVRVNSVHPGGVDTLMANNNQRPREQVNTSYTNVPLQRIGGPEEVAAVSLFLASDDASYMTGAELLVDGGMTIGHYYPGLPGAPG

SEQ ID NO: 130 - protein, 259 aa, Novosphingobium sp.
MGKLAGKVAIITGGARGMGAATSRLFVAEGAKVAIADVLEEDGAALAAELGDAARFYKLDVTSEDDWARVVAAAEADLGPIDALVNNAGILMFKSILETTKADYERVLGVNLVGEFLGIKAVAPGMIARSKGPIVNISSVDGMKGANSLVAYASSKWGVRGLTKVAAMELGHKGVRVNSVHPGGVDTVMSNHTGAPRAEVDKGYANVPMQRIGGPEEVAAASAFLASDESSYCHGTEIVVDGGMTVGTYYMGFPGSPGM

SEQ ID NO: 131 - protein, 259 aa, Novosphingobium sp.
  source 1..259: note = strain Fuku2-ISO-50
MGRLDGKVAIITGGARGMGEATSRRFVAEGAKVAIADVLEDRGQALAAELGDAARFWKLDVTSEDAWTRVVAEVEAAFGGIDVLVNNAGILLFKSILDTTKADYERVLGVNLVGEFLGVRSVAPGMIARGRGSIVNISSVDGMKGANSLVAYASSKWGVRGLTKVAAMELGHKGIRVNSVHPGGVDTVMSNHDGRARSDVDKAYDNVPLQRIGGPEEVAAASLFLASDDASYLNGAEIVVDGGMTVGTYYHGFPGAPGV

SEQ ID NO: 132 - protein, 259 aa, Sphingobium sp.
  source 1..259: note = strain OAS761
MGRLDGKVAIITGGARGMGAATSRLFATEGAKVAIADLLDEAGAALAEELGDAARFYRLDVTDEEAWANMVAAVEADLGPVDILVNNAGILLFRTLLETTRAEYEKVLEVNLVGQFLGLKAVAPGMIARGGGAVVNISSVDGMKGANGLAAYSSSKWGVRGLTRVAALELGHKGIRVNSVHPGGVDTVMTNHDGRSRDAVNERFGNIPLQRVGAPEEVARASLFLASDDASYLSGAEIAVDGGMMTGQYYEGYPGAPGV

SEQ ID NO: 133 - protein, 256 aa, unidentified
  source 1..256: note = Pseudomonadota bacterium
RLAGKVAIITGGARGMGAATSRLFVAEGAKVAIADMLDAEGTTLAAELGPNARFWKHDVTSEAAWATLVAEVEQAFGGVDVLVNNAGIIIFKSLLDTSADEYKRVLDVNLVGEFLGIKAVAPGMIARGKGSIVNISSVDGMKGANCLAAYASSKWGVRGLTKVAAMELGHKGVRVNSVHPGGVDTVMSNHSNAPRSEVDKAYGNVPLQRIGGPEEVAAASLFLASDDASYIHGTEVVVDGGMTTGAYYTGFPGSPG

SEQ ID NO: 134 - protein, 259 aa, unidentified
  source 1..259: note = Novosphingobium aerophilum
MGRLDGKVAIVTGGARGMGAATVRRFVAEGAKVAIADVLDEAGQALAAELGEAAAFWHLDVTSEQAWAEVVAAVEARFGGIDVLVNNAGVLLFKSLLDTTKADYERVLGVNLVGEFLGIKAVAPGMIARGKGSIVNISSVDGMKGANSLVAYASSKWGVRGLTKVAAMELGHKGVRVNSVHPGGVDTIMSNHDNRPRTEVDKAYTNVPMQRIGGPEEIAAASLFLASDDASYLNGAEIVVDGGMIVGTYYDGFPGAPGV

SEQ ID NO: 135 - protein, 259 aa, unidentified
  source 1..259: note = Novosphingobium aerophilum
MGRLDGKVAIVTGGARGMGAATVRRFVAEGAKVAIADVLDEAGQALAAELGEAAAFWHLDVTSEPAWAEVVAAVEARFGGIDVLVNNAGVLLFKSLLDTTKADYERVLGVNLVGEFLGIKAVAPGMIARGKGSIVNISSVDGMKGANSLVAYASSKWGVRGLTKVAAMELGHKGVRVNSVHPGGVDTIMSNHDNRPRTEVDKAYTNVPMQRIGGPEEIAAASLFLASDDASYLNGAEIVVDGGMIVGTYYDGFPGAPGV

SEQ ID NO: 136 - protein, 257 aa, Sphingobium vermicomposti
KLQGKTAIITGGARGMGAATSRLFAAEGAKVAIADLLDAEGAALAAELGDAARFYHHDVTSQDDWAKLVAAVEADLGPVDILVNNAGILLFRTLFETTLADYQKVLNVNLIGEFLGIKAVGPGMVARGKGAIVNISSVDGMKGANGLGAYASSKWGVRGLTRVAAMELGHKGVRVNSVHPGGVDTIMSNQEGRSRDQVDQGYGNIPLQRIGAPEEVAKASLFLASDDASYLTGAEIVVDGGMVIGQYYEGFPGAPGV

SEQ ID NO: 137 - protein, 259 aa, unidentified
  source 1..259: note = Novosphingobium jiangmenense
MGKLEGKVAIITGGARGMGAATSRRFVAEGAKVAIADVLEAEGAALAAELGDAARYYKLDVTSEESWASVVAAVEADLGPVGVLVNNAGILMFKSLLETTKADYERVLGVNLVGEFLGIKAVAPGMIARGQGSIVNVSSVDGMKGANSLVAYASSKWGVRGLTKVAAMELGHKGVRVNSVHPGGVDTVMSNHSGVARSELDKSYGEVPLQRIGAPEEVAAASAFLASDDASYLHGAEIVVDGGMTVGTYYHGFPGSPGM

SEQ ID NO: 138 - protein, 259 aa, Novosphingobium sp.
  source 1..259: note = strain BL-8H
MGRLEGKVAIVTGGARGMGAATCRRFVAEGAKVAIADVLDEAGRALAAELGDAAGFWHLDVTSEADWAAVVQGVEERFGPVNVLVNNAGILHFAGLLDTSLADYRRVLDVNLVGEFLGIKAVAPGMVARGKGAIVNVSSVDGMKGANGLSAYASSKWGVRGLTRVAAMELGHKGVRVNSVHPGGVDTVMTNHDGSPRDKVDKRFGNIPLQRVGAPEEVAAATVFLASDEASYLAGAEIVVDGGMIVGAYYQGMPGAPGM

SEQ ID NO: 139 - protein, 256 aa, Sphingobium sp.
  source 1..256: note = strain ZW T5_29
KLQGKTAIITGGARGMGAATSRLFAAEGAQVAIADLLDAEGAALAAELGDAARFYHHDVTSEEDWAKLVAAVEADLGPVDILVNNAGILLFRTLFDTTLADYQRVLNVNLVGEFLGIKAVGPGMVARGKGAIVNISSVDGMKGANGLGAYCSSKWGVRGLTRVAAMELGHKGVRVNSVHPGGVDTVMTNQEGRSRDQVDQAYGNVPLQRVGAPEEVAKASLFLASDDSSYLTGAEIVVDGGMVVGQYYMGFPGAPG

SEQ ID NO: 140 - protein, 259 aa, Novosphingobium sp.
  source 1..259: note = strain BL-8A
MGRLEGKVAIVTGGARGMGAATCRRFVAEGAKVAIADVLDDAGKALAEELGEAAMFCHLDVTSEDNWAEVVRGVEARFDPVDVLVNNAGILHFATLLDTSLADYRRVLDVNLVGEFLGIKAVAPGMVARGKGAIVNVSSVDGMKGANGLSAYASSKWGVRGLTRVAAMELGHKGVRVNSVHPGGVDTVMTNHEGAARDKVNERFGNIPLQRVGAPEEVAAATVFLASDEASYLAGAEIVVDGGMIVGAYYEGVPGAPGM

SEQ ID NO: 141 - protein, 259 aa, Novosphingobium sp.
MGKLDGKVAIITGGARGMGEATSRRFVAEGAKVAIADVLVEQGEALAAELGVNARFWKLDVTNEDEWTSVIAAVEAAFGGIDVLVNNAGILMFKSILETTKADYERVLGVNLVGEFLGVRSVAPGMIARGRGSIINLSSVDGMKGANSLVAYASSKWGVRGLTKVAAMELGHKGIRVNSVHPGGVDTIMSNHNGRARSELDPTYANVPLQRIAGPEEVAAASLFLASDDASYVNGAEIVVDGGMTTGTYYLGFPGAPGV

SEQ ID NO: 142 - protein, 259 aa, unidentified
  source 1..259: note = Sphingomonadaceae bacterium
MGRLDGKVVIITGGARGMGEATSRLFAAEGARVAICDVLEEQGVALAAELGGDARFYRLDVTDENGWAEVVAAVEAEIGPVDTLVNNAGVLLFSSLLETSLADYERVLRINLVGEFLGIKAVGPGMVARGKGAIVNISSVDGMKGANGLAAYASSKWGVRGLTKVAAMELGHKGVRVNSVHPGGVDTIMSNYAAAPREEVDKQYGNVPLQRIGGPEEIAQASLFLASDDASYIHGAELVVDGGMIVGQYYDGMPGAPGV

SEQ ID NO: 143 - protein, 259 aa, Novosphingobium sp.
  source 1..259: note = strain AAP83
MSMLSGKVAIITGGARGMGAATSRLFVQHGAKVAIADVLDEAGEALALELGDSARFFKLDVTSEESWAQVVADVEGTFGPVDALVNNAGILMFKSILETTKADYERVLGVNLVGEFLGVKAVAPGMIARGKGAIVNISSVDGMKGANGLVAYASSKWGVRGLTKVAAMELGHKGIRINSVHPGGVDTIMSNHNNAARDELDKAYGNVPLQRIGGPEEVAAASAFLVSDAASYIHGAEIVVDGGMTVGTFYMGMPGAPGM

SEQ ID NO: 144 - protein, 259 aa, Sphingobium sp.
MGRLDGKVAIITGGARGMGAATSRLFVAEGAKVAIADLLDEAGAALAAELGEAARFYRHDVTSEEEWAALVAAVEADLGPVDILVNNAGILLFRTLLETTRAEYEKVLTVNLVGEFLGIKAVAPGMIARGKGSIVNISSVDGMKGANGLAAYSSSKWGVRGLTRVAALELGHRGIRVNSVHPGGVDTVMTNQDGRSRDAVNERFGNIPLQRVGGPEEVAKATLFLASDDASYLAGAEIAVDGGMMTGQYYEGFPGAPGV

SEQ ID NO: 145 - protein, 259 aa, Novosphingobium sp.
  source 1..259: note = strain ERN07
MGKLAGKVAIITGGARGMGAATSRLFVAEGAKVAIADVLEDDGAALAAELGEAARFYKLDVTDEGDWTRVVAAAEADLGPVDVLVNNAGILMFKSILETTKADLERVLGVNLVGEFLGIKAVAPGMIARGKGAIVNVSSVDGMKGANSLVAYASSKWGVRGLTKVAAMELGHKGIRVNSVHPGGVDTVMSNHSGAPRAEIDKSYGNVPMQRIGGPEEVAAASAFLASNEASYCHGTEIVVDGGMTVGTYYMGFPGSPGM

SEQ ID NO: 146 - protein, 256 aa, Novosphingobium sp.
  source 1..256: note = strain FKTRR1
RLAGKVAIVTGGARGMGAATSRLFVAEGAKVAIADMLDAEGAALAAELGENARFWKHDVTDEAAWATLVAEVEAAFGGVDVLVNNAGIIIFKGMLDTTHDDFKRVLDVNLVGEFLGIKAVAPGMIARGKGAIVNISSVDGMKGANCLVAYASSKWGVRGLTKVAAMELGHKGVRVNSVHPGGVDTIMSNHGSVSRAELNTTYSHVPLQRIGGPEEVAAASLFLASDDASYIHGTEVVVDGGMTTGVYYHGFPGAPG

SEQ ID NO: 147 - protein, 259 aa, unidentified
  source 1..259: note = Novosphingobium
MGKLEGKVAIVTGGARGMGAATCRRFVAEGARVAIADVLEAEGAALAAELGAAARFFRLDVTSEENWAEVVAAVEAGLGPVDVLVNNAGILMFKSLLETTKADYERVLGVNLVGEFLGIKAVAPGMIARGKGAIINISSVDGMKGANSLVAYASSKWGVRGLTKVAAMELGHKGIRVNSVHPGGVDTVMSNHTGAPREEVDKGYANVPLQRIGGPEEVAAASAFLASDDASYVHGAEIVVDGGMTVGTYYMGFPGSPGM

SEQ ID NO: 148 - protein, 257 aa, Hydrocarboniphaga sp.
RLQDKVAIITGGARGMGAATVRMFVAEGAKVIITDVLETEGRALAAELGEQAAFIRHDVSDEAAWRQVLAFAVERYGRVDVLVNNAGILVFRSLLDTSKADFERVLSVNLTGTFLGLHTIAPHMIAKGRGSIVNISSIDGMKGANSLAAYASSKWGIRGLTKVAAMELGHRGLRVNSVHPGGIDTAMTNPNGVAREVLDPHYTMVPMQRTGAPEEVAAATLFLASDEASYINGAEIAVDGGLVIGQYYQGFPGAPGV

SEQ ID NO: 149 - protein, 259 aa, Sphingobium jiangsuense
MDRLKGKVAIITGGARGMGAATSRLFAAEGAKVAITDLLEDAGAALAAELGEAARFYRHDVSSEADWAAVVGAVEADLGPVDILVNNAGILLFRTLLDTSLEEYRRVLDVNLTGEFLGIKAVAPGMIARGKGSIVNISSVDGMKGANGLAAYSSSKWGVRGLTRVAALELGHRGIRVNSVHPGGVDTVMTNYEGAAREKVNERFGNIPLQRVGGPEEVARATLFLASDEASYLAGAEIAVDGGMLTGHYYTGMPGAPGV

SEQ ID NO: 150 - protein, 259 aa, Nevskia soli
MARLDGKVAIITGGARGMGGATSRLFAAEGAKVVIADVLDKEGAELAQELKGSAIFQHHDVTDEASWANVVAKAIATFGKVDILVNNAGILLFKTLLDTSKADYERVLGVNLMGAFLGIKAVAPHMIERGNGSIVNVSSVDGMKGANSLGAYSSSKWGLRGLTRVAAMEFGHKGVRVNSIHPGGIDTAMGNPYSENRTEVNKRYTMVPLQRVGDPIEAARTSLFLASDDSSYLCGAEIAVDGGMLTGQYYVGFPGAPGV

SEQ ID NO: 151 - protein, 259 aa, Nevskia sp.
MARLDGKVAIITGGARGMGGATSRLFAAEGAKVVIADVLDKEGAELAQELKGSAIFQHHDVTDEASWANVVAKAIATFGKVDILVNNAGILLFKTLLDTSKADYERVLGVNLMGAFLGIKAVAPHMIERGNGSIVNVSSVDGMKGANSLGAYSSSKWGLRGLTRVAAMEFGHKGVRVNSIHPGGIDTAMGNPYSENRTEVNKRYTMVPLQRVGDPIEAARTSLFLASDDSSYLCGAEIAVDGGMLTGQYYVGFPGAPGI

SEQ ID NO: 152 - protein, 258 aa, Hydrocarboniphaga daqingensis
GRLHNKIAIITGGARGMGAATVRLFVEHGAQVLFTDVLDEAGQALAAELGAQATFMHHDIADEAAWQQVLDAAMTRFGRVDVLVNNAGILLFRTLLETTKADYERILSVNLTGMFLGIRTIAPQMIANGGGSIVNISSVDGMKGANGLAAYASSKWGARGLTKVAAMELGHRGIRVNSVHPGGIDTVMTNPQGVSREQLDPMYAMVPQQRAGAPSEVAATTLFLASDDASYINGAEIAVDGGLVVGQYYVGYPGAPGV

SEQ ID NO: 153 - protein, 259 aa, Novosphingobium sp.
  source 1..259: note = strain PhB165
MGRLEGKVAIVTGGARGMGAATCRLFVAEGAKVAIADVLDEAGAALAAELGDVAGFWHLDVTSEADWAAVVRDVEARFGPVDVLVNNAGILHFAGLLDTTLADYRRVLDVNLVGEFLGIKAAAPGMVARGKGAIVNVSSVDGMKGANGLSAYASSKWGVRGLTRVAAMELGHKGVRVNSVHPGGVDTVMTNHDGSPRDKVDERFGNIPLQRVGAPEEVAAATVFLASDEASYLAGAEIVVDGGMIVGAYYQGMPGAPGM

SEQ ID NO: 154 - protein, 259 aa, Novosphingobium sp.
MGRLSGKVAIITGGARGMGAATCRLFVEQGAKVVIADVLDDAGAALAAELGESARFLKLDVTNEENWADVVTEAEATLGPVDALVNNAGILMFKSILETTKADFERVLGVNLVGELLGIKAVAPGMIARGRGSIINVSSVDGMKGANGLVAYASSKWGVRGLTKVAAMELGHKGVRVNSVHPGGVDTIMSNHNDSSREELNKSYSNVPLQRIGGPEEIAAASAFLASDDASYIHGAEIVVDGGMTVGTYYMGFPGSPGM

SEQ ID NO: 155 - protein, 259 aa, Novosphingobium sp.
  source 1..259: note = strain SL115
MGKLTGKVAIITGGARGMGAATSRLFVAEGAKVAIADVLEEDGAALAAELGEAARFYKLDVTSEDDWARVVATAQADLGPVDALVNNAGILMFKSILETSKADYERVLGVNLVGEFLGIKAVAPGMISRGKGAIVNVSSVDGMKGANSLVAYASSKWGVRGLTKVAAMELGHKGIRVNSVHPGGVDTVMSNHTGAARAEVNKGYANVPMQRIGGPEEVAAATAFLASDESSYCHGTEIVVDGGMTVGTYYMGFPGSPGM

SEQ ID NO: 156 - protein, 259 aa, unidentified
  source 1..259: note = unclassified Novosphingobium, Novosphingobium sp. 32-60-15
MGRLSGKVAIITGGARGMGAATCRLFVEQGAKVVIADVLDDAGAALAAELGESARFLKLDVTNEENWADVVTEAEATLGPVDALVNNAGILMFKSILETTKADFERVLGVNLVGELLGIKALAPGMIARGRGSIINVSSVDGMKGANGLVAYASSKWGVRGLTKVAAMELGHKGVRVNSVHPGGVDTIMSNHNDSSREELNKSYSNVPLQRIGGPEEVAAASAFLASDDASYIHGAEIVVDGGMTVGTYYMGFPGSPGM

SEQ ID NO: 157 - protein, 259 aa, unidentified
  source 1..259: note = Spongiibacteraceae bacterium
MARLQGKVAVITGGARGMGAATSRLFVAEGAKVVIADVLDQEGRQLAEELAGKAIFVHHDVTDEASWNSVIGAAKNAFGPIDILVNNAGILLFKSIAQTTKADYERVLDVNLVGTFLGVKIVGEHMTANKKGSIVNISSVDGMKGANSLAAYSSSKWGVRGLTRVAAMEFGHQGVRVNSVHPGGINTVMANPNAVDRSEIDKQYGHVPLQRAADAEEVARVTLFLASDDASYVCGAEIAVDGGMLTGHYYHGFPGAPGV

SEQ ID NO: 158 - protein, 259 aa, unidentified
  source 1..259: note = Chloroflexota bacterium
MTRLKEKVAIITGGARGMGSATSRIFAEEGARVIIADVLDEEGEKLAQELGDSVIFCHHDVTVEKSWQAITSKAETEFGKIDVLVNNAGVLLFKSIADTEKEEFERVLGVNLVGAFLGIKTVGPRMVAAKQGSIVNISSVDGMKGANGLAAYCSSKWGIRGLTRVAAMELGHHGVRVNSVHPGGVNTIMSNPTFAVREDVDKNYTHVPLQRIGEPEEVARATVFLASDDASYLCGAEIAVDGGMMTGHYYQGLPGAPGV

SEQ ID NO: 159 - protein, 258 aa, Nevskia sp.
MARLAGKVAIVTGGARGMGGATSRLFAAEGAKVVIADVLDKEGAALAQELGAATMFEHLDVSDEANWAQVVEKTIARFGQVDVLVNNAGILLFKTLLDTSKAEYERIIQVNLMGQFLGIKAVAPHMIARGKGSIVNVSSTDGMKGANGLGAYSSSKWGVRGLTRVAAMELGHKGVRVNSIHPGGIDTAMGNPYAEGKAEVNKRYTWVPLQRVGEPSEAAHCSLFLASDEASYVCGAELAVDGGMLTGQYYVGFPGAPG

SEQ ID NO: 160 - protein, 258 aa, Nevskia sp.
MARLDGKVAIITGGARGMGGATSRLFAAEGAKVVIADVLDKEGAELAAELKGSAIFQHHDVSDEASWASVVAKAIATFGQVDILVNNAGILLFKTILETSKAEYERVIGVNLMGAFLGIKAVAPHMIGRGKGSIVNVSSTDGMKGANGLGAYSSSKWGLRGLTRVAAMELGHKGVRVNSIHPGGIDTAMGNPYGEAREEVNKRYTFVPLQRVGEPSEAAHCSLFLASDDASYVCGAEIAVDGGMLTGPYYVGFPGAPG

SEQ ID NO: 161 - protein, 259 aa, Thauera sp.
MIRLDNKIAIITGGARGMGAATSRLFVEAGARVLIADLLDEQGTALASELGEAAHFQHHDVTDPASWAALVKDTVARWGQIDVLVNNAGVLLFRTLLDTRPEEFDKVLRVNLMGSFLGIQAVAPHMIERGQGAIVNVSSVDGMKGANGLGAYCSSKWGLRGLTRVAALELGHRGVRVNSVHPGGIDTVMSNPRALEREQIDHSYANLPLQRIGAPEEVARTSLFLASDAASYLCGAEIAVDGGMTAGQYYPGLPGAPGV

SEQ ID NO: 162 - protein, 259 aa, Novosphingobium sp.
MGRLEGKVAIVTGGARGMGAATCRRFVAEGAKVAIADVLDEAGAALAAELGEAAGFWHLDVTSEADWAAVVRGVEGRFGPVDVLVNNAGILHFAGLLDTTLADYRRVLDVNLVGEFLGIKAVAPGMVARGKGAIVNVSSVDGMKGANGLSAYASSKWGVRGLTRVAAMELGHKGVRVNSVHPGGVDTVMTNHDGSPRDKVDERFGNIPLQRVGAPEEVAAATVFLASDEASYLAGAEIVVDGGMIVGAYYQGMPGAPGM

SEQ ID NO: 163 - protein, 259 aa, Novosphingobium sp.
MGRLSGKVAIITGGARGMGAATCRLFVEQGAKVVIADVLDDAGAALAAELGESARFLKLDVTNEENWAHVVAEAEAILGPIDALVNNAGILMFKSILETTKADFERVLGVNLVGELLGIKAVASGMIARGRGSIINVSSVDGMKGANGLVAYASSKWGVRGLTKVAAMELGHKGVRVNSVHPGGVDTIMSNHNDSSREDLNKAYSNVPLQRIGGPEEVAAASAFLASDDASYIHGAEIVVDGGMTVGTYYMGFPGSPGM

SEQ ID NO: 164 - protein, 258 aa, Nevskia sp.
MARLTGKVAIVTGGARGMGAATSRLFAQEGAKVVIADVLDQDGAKLAQEIGANAIFEHLDVSDEANWARVVETAIAKFGQVDILINNAGILLFKTLLETSKADYERVLQVNLAGQFLGIKAVAPHMIGRGKGSIVNVSSTDGMKGANGLGAYSSSKWGVRGLTRCAAMELGHKGVRVNSIHPGGIDTAMGNPYAEAKAEVNKRYTWVPAQRVGEPSEAAHCSLFLASDDASYVCGAELAVDGGMLTGTYYVGFPGAPG

SEQ ID NO: 165 - protein, 259 aa, Nevskia sp.
MARLQGKVAIVTGGARGMGAATSRLFAAEGARVVIADVLESEGAALAQEIGADAVFQRHDVSDEASWQVTVRRALDSFGGVDVLINNAGVLLFKTLLDTTKADFERVLGINLMGSFLGIKAVAPHMVERGRGSIVNVSSVDGMKAANGIGAYASSKWGLRGLTRAAAMEFGHKGVRVNSIHPGGIDTAMGNPYGESRAEVNKRYTMVPLQRVGEPAEVAKTSLFLASDDSSYLCGAEIAVDGGMLAGFYYTGFPGAPGV

SEQ ID NO: 166 - protein, 258 aa, unidentified
  source 1..258: note = Pseudomonadota bacterium, Rhodocyclaceae bacterium
MQRLQDKVAIITGGARGMGAATARLFATEGARVVITDVLDADGAALATELGGGAVYRHLDVTDENGWRQVVQQTEQDAGRIDILINNAGILMFKSLLEIEKAEFEKVLNINLVGAFLGIRSVAPGMIRQGRGAIVNISSVDGMKGANGLGAYASSKWGLRGLTRVAAMELGHKGIRVNSVHPGGINTIMANPNGAPRSEIDKYYVSVPLQHAGEPEEVAKANLYLASDEAAFVTGAEIAVDGGMVIGRYYEGLPGAPG

SEQ ID NO: 167 - protein, 259 aa, Nevskia sp.
MARLAGKVAIITGGARGMGGATSRLFAQEGAKVVIADVLDQDGARLAQEIGANAIFEHLDVSDEANWARVVDKAIARFGQVDILVNNAGILLFKTLLETSKAEYERVLQVNLMGQFLGIKAVAPHMIGRGKGSIVNVSSTDGMKGANGLSAYSSSKWGVRGLTRCAAMELGHKGVRVNSIHPGGIDTAMGNPYAEAKAEVNKRYTWVPAQRVGEPSEAAHCSLFLASDEASYVCGAELAVDGGMLTGTYYVGFPGAPGI

SEQ ID NO: 168 - protein, 259 aa, unidentified
  source 1..259: note = Chloroflexota bacterium
MDRLQDKVAIVTGGARGMGSATARLFAEEGARVIIADVLDREGEELARQLKGPAVFAHHDVTDEESWQKIVSDTEAQFGKIDVLVNNAGILLFKSIADTEKEEFERVLGVNLVGAFLGIKTVGSRMAAHKKGSIINVSSVDGMKGANGLAAYCSSKWGIRGLTRVAAMELGHRGVRVNSVHPGGINTIMSNPTFAPREAVDKRYTHVPLQRIGDPEEVARATLFLASDDASYLCGAEIAVDGGMLVGHYYQGLPGAPGV

SEQ ID NO: 169 - protein, 259 aa, Solimonas flava
MSRLRNKVAIITGGARGMGAATARLFAAEGAKLVISDVLDDDGARLAAELGERALFVHHDVSDENAWHGVLDRAVAAFGGVDVLVNNAGVLLFRTLAETSKADFERVIDINLVGTFLGVKIVGQHMVGRRHGSIVNISSVDGMKGANGLGAYASSKWGIRGLTRVAAMEYGHKGVRVNSVHPGGIDTAMGNPYAEARAEVNKRYAMVPLQRVGEPDEVARTSLFLASDESSYLCGAEIAVDGGMLTGQYYVGFPGAPGV

SEQ ID NO: 170 - protein, 259 aa, unidentified
  source 1..259: note = Solimonas marina
MSRLRDKVAIITGGARGMGASTARLFVREGARVVISDVLEDDGAALARELGTAARFVAHDVRDEAQWQQVLDTATDVFGPVDVLVNNAGVLVFRTLAETEKSEFERVIDINLLGTFLGVRIVGAHMLARGRGSIVNVSSVDGMKGANGLGAYSASKWGIRGLTRVAAMEYGHRGVRVNSIHPGGIDTAMGNPYSEARAEVNKRYAMVPLQRVGDADEVARTSLFLASDDSSYLCGAEIAVDGGMLTGQYYVGFPGAPGV

SEQ ID NO: 171 - protein, 259 aa, Solimonas variicoloris
MSRLRNKVAIITGGARGMGAATARLFAAEGAKLVISDVLDDDGARLAAELGERALFVHHDVSDENAWYGVLERAVAAFGGVDVLINNAGVLLFRTIAETSKADFERVIDINLVGSFLGVKIVGQHMVGRRHGSIVNISSVDGMKGANGLGAYASSKWGIRGLTRVAAMEYGHKGVRVNSVHPGGIDTAMGNPYAEARAEVNKRYAMVPLQRVGEPDEVARTSLFLASDESSYLCGAEIAVDGGMLTGQYYVGFPGAPGV

SEQ ID NO: 172 - protein, 259 aa, unidentified
  source 1..259: note = Pseudomonadota bacterium
MARLNNKVAIITGGARGMGAATVRLFAAEGAKVVIADVLDADGEKLAAELAGKAIFVHHDVTSEESWSNVIAKTKAAFGEIDVLVNNAGVLLFKTIATTEKSDFERVITINLTGTFLGTKIVGEQMTARGKGSIVNISSADGMKGANGLGAYCASKWGIRGLTKVAAMEYGHKGVRVNSVHPGGIDTAMGNPYAEERAEVNKRYTMVPLQRVGEPNEVAQTSLFLASDDSSYLCGAEIAVDGGMLVGQYYVGFPGAPGV

SEQ ID NO: 173 - protein, 261 aa, unidentified
  source 1..261: note = Nevskiales bacterium
MQRLKDKVAIITGGARGMGAATARLFVENGARVVIADVLDTEGAALADELGGAARFVHHDVTDEASWAGLLQLTTADIGPVDVLVNNAGVLLFKTITATEKSEFERVIGINLVGCFLGTRIVGTHMLANGRGSIVNISSVDGMKAANSLGAYCASKWGVRGFTKVAAMEFGHRGVRVNSVHPGGIDTAMGNPYQQGKAEVNQRYSMVPMQRVGEPEEVARTTLFLASDESSYLCGAEIAVDGGMLCGQYYVGFPGAPGVPD

SEQ ID NO: 174 - protein, 259 aa, unidentified
  source 1..259: note = Stenotrophobium sp.
MTRLKDKVALITGGARGMGAATTRLFAAEGAKVVIADVLDADGAALAKELGGRAIFVHHDVTDEASWANVIAKARSAFGEIDVLVNNAGVLLFKTIAETQKADFEKVISVNLTGTFLGTRLVGAQMVARGKGSIVNISSADGMKGANGLGAYCASKWGIRGLTKVAAMEFGHKGVRVNSVHPGGIDTAMGNPYAQEKAEVNKRYTMVPLQRVGEPDEVARTSLFLASDDSSYLCGAEIAVDGGMLAGQYYVGFPGAPGV

SEQ ID NO: 175 - protein, 259 aa, Novosphingobium sp.
MGKLAGKVAIITGGARGMGAATSRLFVAEGAKIVIADVLEAEGAALAAELGEAARFYKLDVTSEDDWASVVAAAEAELGPVDVLVNNAGVVMFKALLDTTKADFEGVLQVNLVGQFLGLKAVAPGMIARGRGAIVNISSVDGMKGANCLAAYASSKWGVRGLTKVAAMELGHKGIRVNSVHPGGVDTIMSNHTGAPRAELDKKYANVPLQRIGGPDEVAAATAFLASDEASYIHGGEIVVDGGMTVGVYYDGFPGAPGM

SEQ ID NO: 176 - protein, 259 aa, unidentified
  source 1..259: note = Stenotrophobium rhamnosiphilum
MARLNNKVAIITGGARGMGAATVRLFAAEGAKVVIADVLDADGEKLAAELAGKAIFVHHDVTSEESWSNVIAKTKAAFGDIDVLVNNAGVLLFKTIATTEKSDFERVITINLTGTFLGTKIIGEQMTARGKGSIVNISSADGMKGANGLGAYCASKWGIRGLTKVAAMEYGHKGVRVNSVHPGGIDTAMGNPYAEERAEVNKRYTMVPLQRVGEPNEVAQTSLFLASDDSSYLCGAEIAVDGGMLVGQYYVGFPGAPGV

SEQ ID NO: 177 - protein, 259 aa, Solimonas sp.
MSRLRNKVAIITGGARGMGGATSRLFVAEGAKVVIADVLDDDGGKLAAELGDSALFVHHDVSDEASWHALLEKAIAKFGGVDVLVNNAGVLLFKSIAETSLADFERVIDINLVGTFLGVKIVGAHMIGRRHGSIVNISSVDGMKGANSLSAYNASKWGIRGLTKAAAMEYGHKGVRVNSVHPGGIDTAMGNPYSEARAEANKRYTMVPLQRIGEPSEVAYTSLFLASDEASYLCGAEIAVDGGMLTGQYYVGFPGAPGV

SEQ ID NO: 178 - protein, 259 aa, unidentified
  source 1..259: note = Nevskiaceae bacterium
MGRLQDKVAIVTGGARGMGAATARVFVAEGAKVAIADVLDEPGARLARELGTRALFQHHDVSDEASWSRLIEVVEARLGPIDVLVNNAGVLLFKTIAETSRAEFERVLGVNLIGCFLGTRRVGARLVARGRGAIVNVSSVDGMKAANGLGAYCASKWAVRGFTKVAAMEFGHRGVRVNSVHPGGIDTAMGNPYAEPKAELNKRYAMVPLQRVGEPEEVARVSLFLASDEASYLNGAEIAVDGGMLCGQYYLGFPGAPGV

SEQ ID NO: 179 - DNA, 1185 bp, Sphingomonas sp.
  source 1..1185: mol_type = genomic DNA; strain = KC8
atgagcggcatgggcaagttcgactgggcggatccattcctgctggacgatcagctgaccgaagacgaacgcatgatccgcgacaccgccgccgcctatgcgcaggacaagctgcaaccgcgcgtgatcgccgcgttcgccaacgaaaccaccaatcccgaaatcttccgcgagatgggcgcgctcggcctgctcggcccgacgatgccggccgaatatggtggcgtcggcgcatcctatgtcgcctacggccttgtcgcgcgcgagatcgaacgggtggattcgggctatcgttcgatgatgtcggtgcagtcgtcgctcgtgatgtacccgatctacgcttatggatcggaagaacagcgccagaaatatctgcccaagctggcaagtggcgaatggatcggctgtttcggcctgaccgaacccgacgccggttccgaccccgccggcatgttgacccgcgccgaaaagatcgacggcggctaccgcatttcggggtccaagacatggatttcgaacgcgccgatcgccgacgtgttcgtcgtctgggccaaatccgacgcgcatggcggcgcgatccgcggcttcatcctcgacaaggggatgaaggggctgtcggcgcccaagatcgaaggcaagctgagcctgcgcgcatcgatcaccggcatgctgatgatggacggggtggaagtgcgcgaagatgcgctgctgcccaaggtggaagggctgaaagggccgttcggctgcctcaaccgggcgcgctatggcatcagctggggggcgcttggcgcggccgaattctgtttccacgcggcgcgccaatatgggctggatcgcaagcaattcggccggccgcttgccgcgacgcaattgtaccagaagaagctggccgacatgatggccgatatcgcgctggggttgcaggccagtttgcgcgtcggccggctgatggacgaaggccggttcgcccccgaaatggtgtcgatcgtcaagcgcaacaatgtcggcaaggcgctggatatcgcccggcaagcccgcgacatgcacggcggcaacggcatttcaggcgaatatcaggtgatgcgccacgcgatgaacctggaaacggtgaacacctatgaaggtgcgcacgacgtgcacgcgctgatcatgggccgcgcgatcaccgatattccggcgttctga

SEQ ID NO: 180 - protein, 394 aa, Sphingomonas sp.
  source 1..394: note = strain KC8
MSGMGKFDWADPFLLDDQLTEDERMIRDTAAAYAQDKLQPRVIAAFANETTNPEIFREMGALGLLGPTMPAEYGGVGASYVAYGLVAREIERVDSGYRSMMSVQSSLVMYPIYAYGSEEQRQKYLPKLASGEWIGCFGLTEPDAGSDPAGMLTRAEKIDGGYRISGSKTWISNAPIADVFVVWAKSDAHGGAIRGFILDKGMKGLSAPKIEGKLSLRASITGMLMMDGVEVREDALLPKVEGLKGPFGCLNRARYGISWGALGAAEFCFHAARQYGLDRKQFGRPLAATQLYQKKLADMMADIALGLQASLRVGRLMDEGRFAPEMVSIVKRNNVGKALDIARQARDMHGGNGISGEYQVMRHAMNLETVNTYEGAHDVHALIMGRAITDIPAF

SEQ ID NO: 181 - DNA, 924 bp, Sphingomonas sp.
  source 1..924: mol_type = genomic DNA; strain = KC8
tcaggcggcgagcgatgcgtcgcgcgcggcgggctggccgaaaccgatgctgaaatcatggccccacaggctgacggcggcgctttcgaagacgatgtgttcgttccagtcgatgacgcggccgccatagccatattcgatcgcgaaaccgcccggcgacgccatgtagaaggacgtcacttcgtcgttcacatggcggccgagcgtggccatcggccggacaccgtggcgcagcatccgatcctgcgcgcggccgacttcatccatgctgtcgacttcgaccatcagatggacgcagccgctgggtacttcgccttcgaacagcgcgaggctgtggtggcggccattgccgcaatgcaggaagtggatgcgctgcgcggtcgggccgtcggggccatcggtgcggtggacgaggatgtccgacaggccgaagcccatcacgttcgccaggaaggcgcaggtttcgtcgaagcggggtgcgggcagcaccatgtggccaaggccgagatcatcggtgatgaagcgcgaaacgccctgcggggactggaagcgctggaaatcgctgacatagccccaggtgatttcgtggcggttgcccgaaggatcgcggaaccaggccatgtcctgcacatgacgaaaggcgatttccgtgggcgtgccgggcgcaacatcgacgccggcggccgcgagatcggcgagggcggcggtgaaggcggcatggtccgccagttcccagccactggcggcatagcgattggcggtggcgcgctggacgaggatgcggccggcgcgggcatccatcttgagcgccagattgaggccgatcggcatggccatcatgccaaggacgtcttcgccatagcggccccaggctgttggatcgaccgcgtcgatgaccacgtaggatagtgccctgatcttcat

SEQ ID NO: 182 - protein, 307 aa, Sphingomonas sp.
  source 1..307: note = strain KC8
MKIRALSYVVIDAVDPTAWGRYGEDVLGMMAMPIGLNLALKMDARAGRILVQRATANRYAASGWELADHAAFTAALADLAAAGVDVAPGTPTEIAFRHVQDMAWFRDPSGNRHEITWGYVSDFQRFQSPQGVSRFITDDLGLGHMVLPAPRFDETCAFLANVMGFGLSDILVHRTDGPDGPTAQRIHFLHCGNGRHHSLALFEGEVPSGCVHLMVEVDSMDEVGRAQDRMLRHGVRPMATLGRHVNDEVTSFYMASPGGFAIEYGYGGRVIDWNEHIVFESAAVSLWGHDFSIGFGQPAARDASLAA

(19) United States
(12) Patent Application Publication
(10) Pub. No.: US 2026/0176666 A1
(43) Pub. Date: Jun. 25, 2026

(54) SYSTEMS AND METHODS FOR ESTROGEN BIOSENSING

(71) Applicant: BioSens8, Inc., Cambridge, MA (US)

(72) Inventors: Uros Kuzmanovic, Cambridge, MA (US); Connor A. Davis, Somerville, MA (US); Roger J. Charles, JR., Cambridge, MA (US)

(73) Assignee: BioSens8, Inc., Cambridge, MA (US)

(21) Appl. No.: 19/424,599

(22) Filed: Dec. 18, 2025

Related U.S. Application Data
(60) Provisional application No. 63/735,965, filed on Dec. 19, 2024.

(51) Int. Cl. C12Q 1/00; C12Q 1/32; G01N 27/327; G01N 33/74

(52) U.S. Cl. CPC C12Q 1/004; C12Q 1/32; G01N 27/3277; G01N 33/743; G01N 2333/723; G01N 2333/902

(57) ABSTRACT

The present disclosure generally relates to enzyme-based biosensors and, in particular, to biosensors for determining estrogen and other biomolecules. In some cases, the enzymes may be based on dehydrogenases, such as 17-beta hydroxysteroid dehydrogenases. In certain embodiments, the enzyme may include an active site that is able to bind to and oxidize estrogen, or other biomolecules. In some cases, the active site may include certain residues positioned around the active site that are able to specifically interact with estrogen, or other biomolecules. Electrons produced from such oxidation reactions may be determined using an electrode to determine estrogen, e.g., in solution. Other embodiments are generally directed to methods of making or using such biosensors, kits including such biosensors, enzymes for use in such biosensors, etc.

### RELATED APPLICATIONS

This application claims the benefit of U.S. Provisional Patent Application Ser. No. 63/735,965, filed Dec. 19, 2024, entitled “Systems and Methods for Estrogen Biosensing,” by Kuzmanovic, et al., incorporated herein by reference in its entirety.

### GOVERNMENT FUNDING

This invention was made with government support under Grant. No. 2341568 awarded by National Science Foundation. The government has certain rights in the invention.

### REFERENCE TO AN ELECTRONIC SEQUENCE LISTING

The contents of the electronic sequence listing (B173670001US01-SEQ-TC.xml; Size: 221,175 bytes; and Date of Creation: Feb. 25, 2026) are herein incorporated by reference in their entirety.

### FIELD

The present disclosure generally relates to enzyme-based biosensors and, in particular, to biosensors for determining estrogen and other biomolecules.

### BACKGROUND

Currently, there are millions of females of reproductive age in the US with fertility issues. The primary reason for them being unable to become pregnant are ovulatory dysfunctions. Fortunately, serum progesterone and estrogen levels can predict and confirm ovulation with very high specificity. However, there are currently no at-home, quantitative, and blood-based hormone monitors which allow females to assess their ovulatory health. Accordingly, there is an unmet need for such females.

### SUMMARY

The present disclosure generally relates to enzyme-based biosensors and, in particular, to biosensors for determining estrogen and other biomolecules. The subject matter of the present disclosure involves, in some cases, interrelated products, alternative solutions to a particular problem, and/or a plurality of different uses of one or more systems and/or articles.

One aspect is generally drawn to a device. In one set of embodiments, the device comprises a 17-beta hydroxysteroid dehydrogenase, configured to specifically react estradiol to produce estrone and electrons, in electrical communication with an electrode.

In another set of embodiments, the device comprises a 17-beta hydroxysteroid dehydrogenase arising from Caenibius tardaugens, in electrical communication with an electrode.

The device, in yet another set of embodiments, comprises an enzyme in electrical communication with an electrode. In some cases, the enzyme has at least 80% homology to EGO55_02230 (SEQ ID NO: 70), and an active site comprising a first sequence GXXXGXG (SEQ ID NO: 2) starting within +/−5 amino acids from position 13, a second sequence LVNNAG (SEQ ID NO: 3) starting within +/−5 amino acids from position 84, and a third sequence YXXXK (SEQ ID NO: 4) starting within +/−5 amino acids from position 155.

According to still another set of embodiments, the device comprises an enzyme selected from any of SEQ ID NOs. 86-178, in electrical communication with an electrode.

Another aspect is generally drawn to a method. According to one set of embodiments, the method comprises oxidizing estradiol in solution to produce estrone and electrons using a 17-beta hydroxysteroid dehydrogenase, and determining an electrical property of an electrode in contact with the solution to determine the estradiol oxidation.

The method, in another set of embodiments, comprises oxidizing estradiol to produce estrone and electrons using a 17-beta hydroxysteroid dehydrogenase, transferring the electrons to an electrode, and determining the estradiol based on the transfer of electrons to the electrode.

In yet another set of embodiments, the method comprises determining a concentration of estradiol by measuring electron flow from a reaction of estradiol with a 17-beta hydroxysteroid dehydrogenase arising from Caenibius tardaugens.

According to still another set of embodiments, the method comprises determining a concentration of estradiol by measuring electron flow from a reaction of estradiol with an enzyme. In some cases, the enzyme has at least 80% homology to EGO55_02230 (SEQ ID NO: 70), and an active site comprising a first sequence GXXXGXG (SEQ ID NO: 2) starting within +/−5 amino acids from position 13, a second sequence LVNNAG (SEQ ID NO: 3) starting within +/−5 amino acids from position 84, and a third sequence YXXXK (SEQ ID NO: 4) starting within +/−5 amino acids from position 155.

The method, in yet another set of embodiments, comprises oxidizing estradiol in solution to produce estrone and electrons using an enzyme selected from any of SEQ ID NOs. 86-178, and determining an electrical property of an electrode in contact with the solution to determine the estradiol oxidation.

In accordance with still another set of embodiments, the method comprises oxidizing estradiol to produce estrone and electrons using an enzyme selected from any of SEQ ID NOS. 86-178, transferring the electrons to an electrode, and determining the estradiol based on the transfer of electrons to the electrode.

In another set of embodiments, the method comprises determining a concentration of estradiol by measuring electron flow from a reaction of estradiol with an enzyme selected from any of SEQ ID NOs. 86-178.

In another aspect, the present disclosure encompasses methods of making one or more of the embodiments described herein, for example, a sensor for determining estrogen or other biomolecules. In still another aspect, the present disclosure encompasses methods of using one or more of the embodiments described herein, for example, a sensor for determining estrogen or other biomolecules.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates certain estrogen compounds;

FIG. 2 is a schematic reaction for determining an estrogen, in one embodiment;

FIGS. 3A-3B illustrate the DNA and amino acid sequence of EGO55_RS02230 (SEQ ID NO: 1 and SEQ ID NO: 70, respectively), arising from Caenibius tardaugens, according to one embodiment;

FIG. 4 illustrates various sequences useful in an active binding site for a dehydrogenase, in one embodiment;

FIG. 5 illustrates various sequences useful in an active binding site for a dehydrogenase, in another embodiment;

FIG. 6 illustrates various sequences useful in an active binding site for a dehydrogenase, in yet another embodiment;

FIGS. 7A-7E illustrate various enzymatic structures with sequence similarity to EGO55_RS02230 useful for determining estrogen, in certain embodiments;

FIG. 8 illustrates various enzymes useful for determining estrogen, in other embodiments; and

FIGS. 9A-9R illustrate various amino acid sequences for various enzymes, in some embodiments.

### DETAILED DESCRIPTION

The present disclosure generally relates to enzyme-based biosensors and, in particular, to biosensors for determining estrogen and other biomolecules. In some cases, the enzymes may be based on dehydrogenases, such as 17-beta hydroxysteroid dehydrogenases. In certain embodiments, the enzyme may include an active site that is able to bind to and oxidize estrogen, or other biomolecules. In some cases, the active site may include certain residues positioned around the active site that are able to specifically interact with estrogen, or other biomolecules. Electrons produced from such oxidation reactions may be determined using an electrode to determine estrogen, e.g., in solution. Other embodiments are generally directed to methods of making or using such biosensors, kits including such biosensors, enzymes for use in such biosensors, etc.

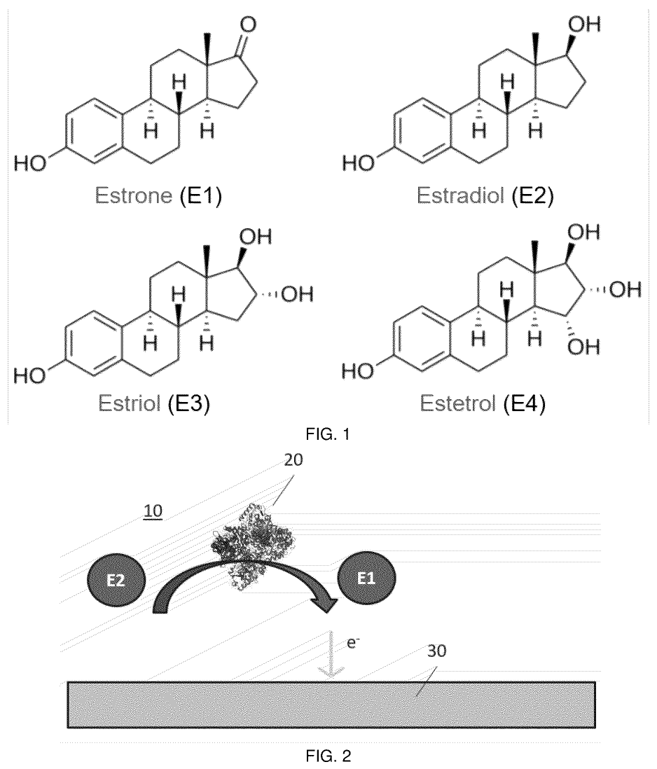

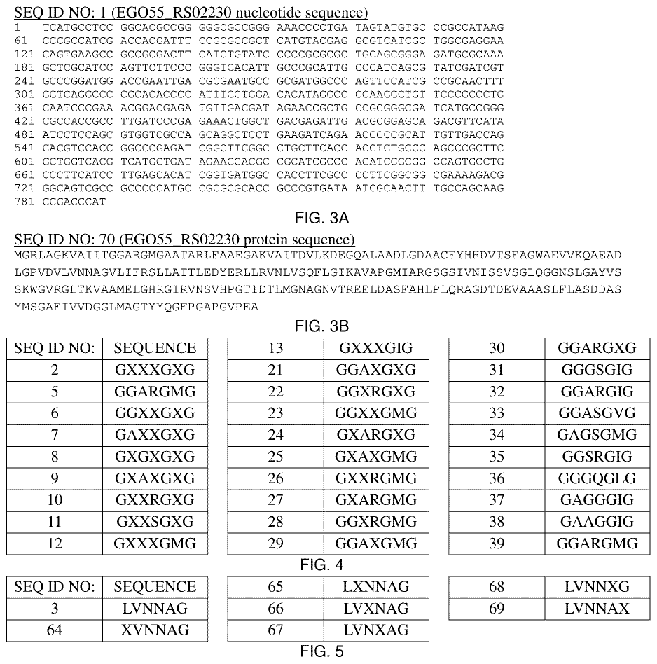

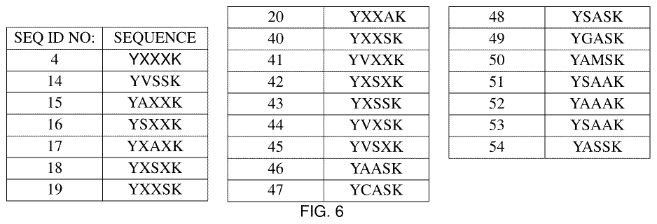

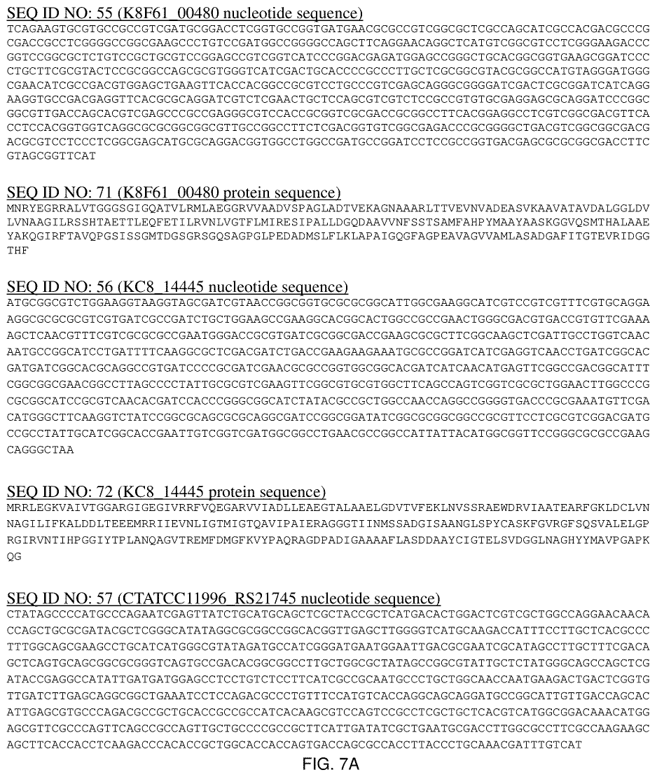

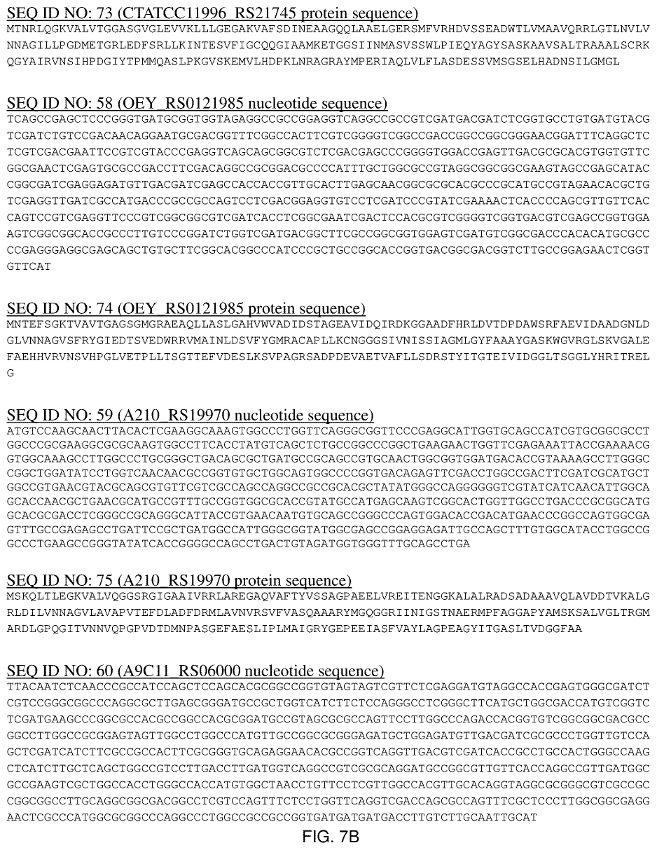

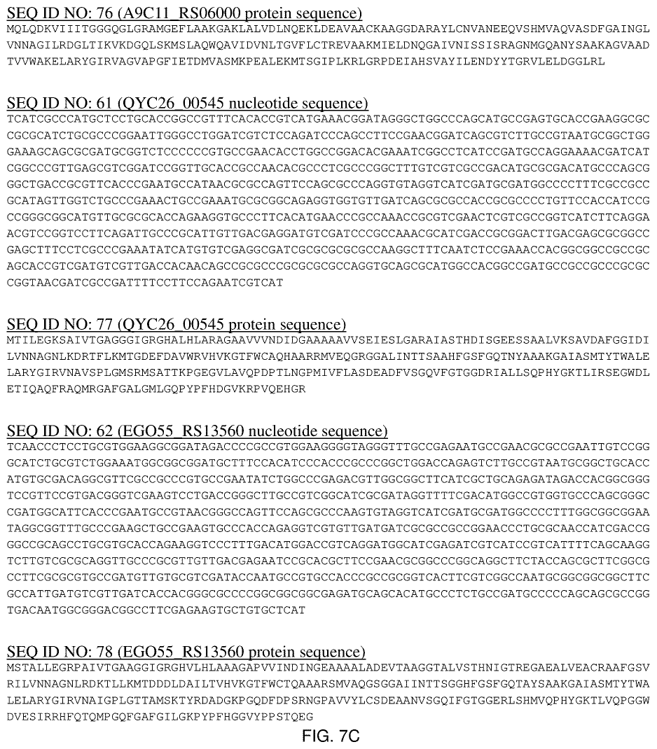

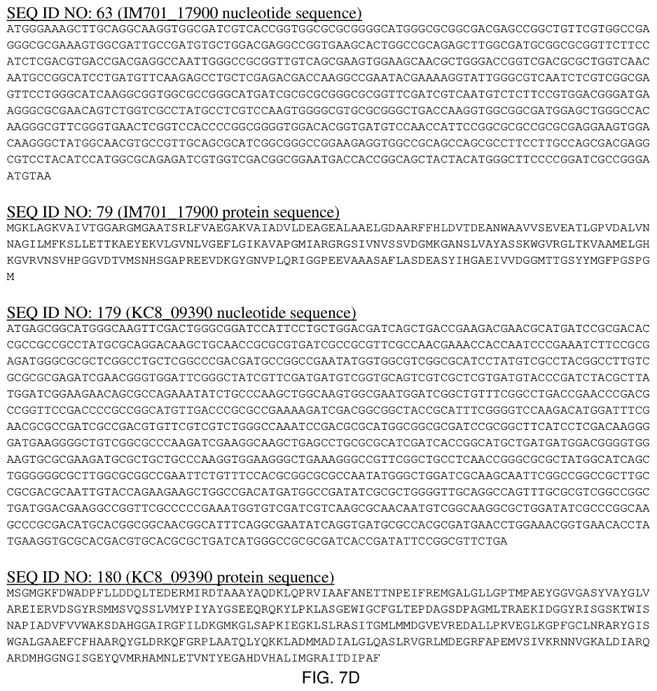

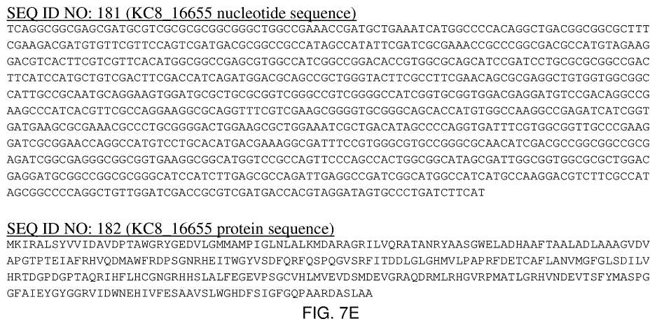

For example, certain aspects are generally directed to various enzymes that are able to bind to and oxidize estrogen, or other biomolecules. Estrogen is a family of sex hormones that are responsible for the development and regulation of the female reproductive system and secondary sex characteristics. The estrogen family includes estrone (abbreviated as E1), estradiol (E2), estriol (E3), and estetrol (E4). See, e.g., FIG. 1. In certain embodiments, the enzyme may oxidize estradiol (E2) to estrone (E1), producing an electron in the process, e.g., which can be determined by the electrode, e.g., as a current or electron flow, i.e.:

$$E 2 \text{‐‐>} E 1 + {e}^{-} .$$

In some oxidation reactions, electrons may be produced that can be sensed, e.g., with an electrode, to determine amounts of estrogen that may be present.

One non-limiting example reaction is shown schematically in FIG. 2. In this figure, in solution 10, an enzyme 20 (e.g., a dehydrogenase or other oxidase) is able to react with E2, converting it to E1, and producing an electron (e), which may be determined by an electrode 30. However, it should be understood that a variety of techniques may be used to transfer the electron to the electrode, and that FIG. 2 is just one non-limiting example. For instance, as discussed herein, electron carriers, redox mediators, or the like may be used to facilitate electron transfer, e.g., to an electrode.

The solution may include a sample taken from a subject, e.g., a blood or other sample, that is suspected of containing estrogen. The subject may be human or non-human. The enzyme may react with the estrogen, e.g., oxidizing it to produce electrons, which can be determined using an electrode. Based on the flow of electrons (e.g., determined as a voltage or current, etc. by an electrode), the amount of estrogen present in the sample may be determined.

A variety of enzymes can be used in various embodiments. A variety of enzymes are described in more detail below. For example, in certain cases, the enzyme may be a dehydrogenase, such as a 17-beta hydroxysteroid dehydrogenase. Such hydroxysteroid dehydrogenases are produced by a variety of organisms. It has been found, however, that not all 17-beta hydroxysteroid dehydrogenases can be used, and in some cases, 17-beta hydroxysteroid dehydrogenases having specific active sites able to recognize estradiol can be surprisingly effective at reducing estradiol to estrone. These include the 17-beta hydroxysteroid dehydrogenase arising from Caenibius tardaugens, and similar enzymes, e.g., those having at least 80% homology. In addition, in some embodiments, a 17-beta hydroxysteroid dehydrogenase may include an active site defined by a first sequence GXXXGXG (SEQ ID NO: 2) starting within +/−5 amino acids from position 13 of the dehydrogenase, and/or a second sequence LVNNAG (SEQ ID NO: 3) starting within +/−5 amino acids from position 84, and/or a third sequence YXXXK (SEQ ID NO: 4) starting within +/−5 amino acids from position 155. (It should be understood that each “X” in any sequences herein may each independently be any suitable amino acid, and different X's within a particular sequence may independently be the same or different.) As discussed herein, these may be able to bind to the estradiol structure and oxidize it to form estrone. Other example active sites and/or dehydrogenases that can be used are also discussed in more detail herein.

The above discussion is a non-limiting example of one embodiment of the present disclosure that can be used to determine estrogen using a dehydrogenase, such as a 17-beta hydroxysteroid dehydrogenase. However, other embodiments are also possible. Accordingly, more generally, various aspects are directed to various systems and methods for determining estrogen and other biomolecules.

For example, some aspects are generally directed to systems and methods for determining estrogen and other biomolecules, e.g., arising from a subject, such as a female subject. For example, the subject may be human, or another non-human subject. Examples of subjects include, but are not limited to, a mammal such as a cow, sheep, goat, horse, rabbit, pig, mouse, rat, dog, cat, a primate (e.g., a monkey, a chimpanzee, etc.), or the like.

The sample may be taken from the subject. Exemplary samples include, but are not limited to, serum, plasma, cell lysate, milk, saliva, vitreous fluid, and other secretions, synovial fluid, peritoneal cavity fluid, lacrimal fluid, and tissue homogenate. In some embodiments, the sample is a bodily fluid, including interstitial fluid, sweat, blood, cerebrospinal fluid (CSF), plasma, whole blood, serum, synovial fluid, saliva, vaginal lubrication, breast milk, amniotic fluid, urine, phlegm tears, saliva, lymph, peritoneal intracellular fluid, or an original tissue. In some cases, the sample can be in various forms including, but not limited to, a liquid, frozen, chilled, lyophilized sample, etc.

The sample may be taken from the subject at any suitable time or frequency. In some cases, only a single sample is taken from a subject. In certain embodiments, more than one sample may be taken from a subject, e.g., on a regular or irregular basis, or a periodic or random basis. For instance, a sample may be taken from a subject once a week, once a day, twice a day, every 12 hours, every 9 hours, every 6 hours, every 3 hours, every 1 hour, etc. In some cases, samples may be taken from the subject continuously, or at a relatively high frequency (e.g., with a period of less than 1 hour, less than 30 minutes, less than 15 minutes, less than 10 minutes, less than 5 minutes, less than 3 minutes, less than 1 minute, less than 30 seconds, less than 15 seconds, less than 10 seconds, less than 5 seconds, etc.).

In some embodiments, the estrogen may be determined through a redox reaction. For example, as discussed above, estrogen may be determined by oxidizing estradiol (E2) to estrone (E1), e.g., using an enzyme. In a redox reaction, electrons may be transferred from one molecule, compound, molecular group, etc. to another. The transfer of electrons can be determined, e.g., using an electrode, such as described herein. In some cases, such reactions may be catalyzed using an enzyme, such as a dehydrogenase (for example, a 17-beta hydroxysteroid dehydrogenase). The dehydrogenase may catalyze a redox reaction using a substrate (e.g., estradiol), resulting in the generation or utilization of electrons, e.g., electron transfer.

In some cases, the device may be an amperometric device, e.g., a device able to determine the current between electrodes. In some cases, the device may be chronoamperometric, e.g., a device able to determine the current between electrodes as a function of time. Other potentiometric techniques may be used in other embodiments. Non-limiting examples include chronoamperometry, intermittent pulse amperometry, double potential-step amperometry, chronopotentiometry (open circuit potentiometry), transient potentiometry, cyclic voltammetry, differential pulse voltammetry, square wave voltammetry, alternating-current voltammetry, fast scan voltammetry, staircase voltammetry, electrochemical impedance spectroscopy (potentiometric), electrochemical impedance spectroscopy (galvanometric), and/or other potentiometrically driven conductivity or impedance measurements used by organic electrochemical transistors (OECTs). Such determinations (e.g., of current, voltage, etc.) may be qualitative and/or quantitative, in various embodiments.

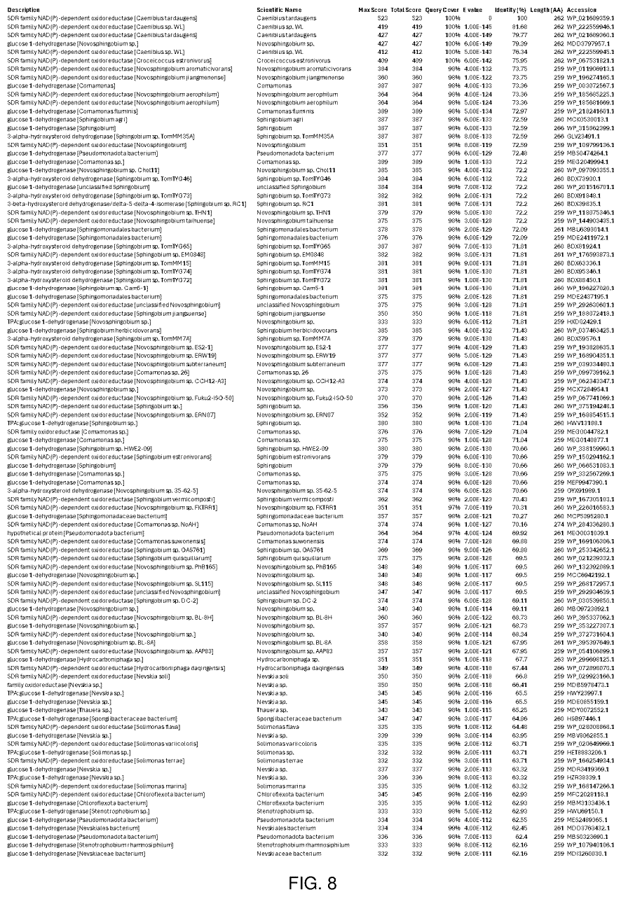

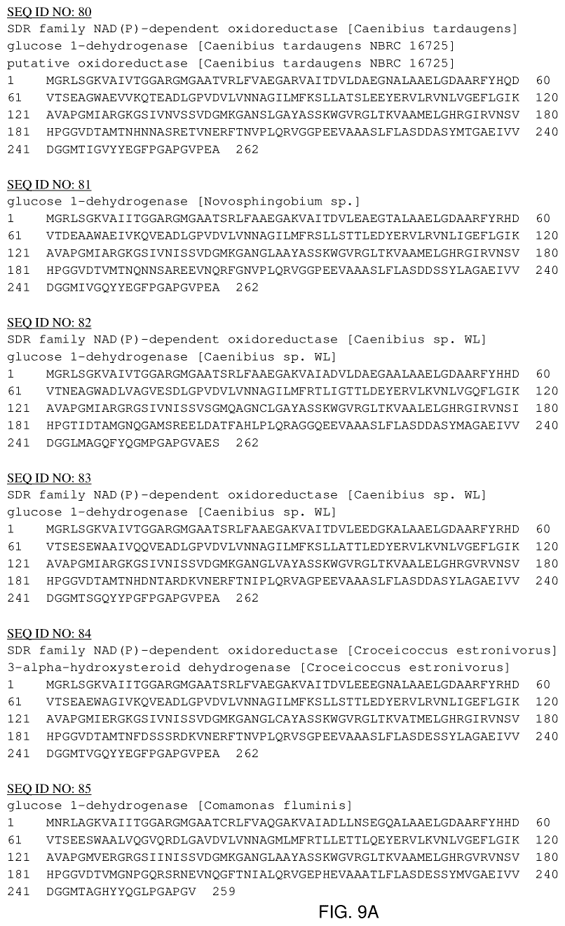

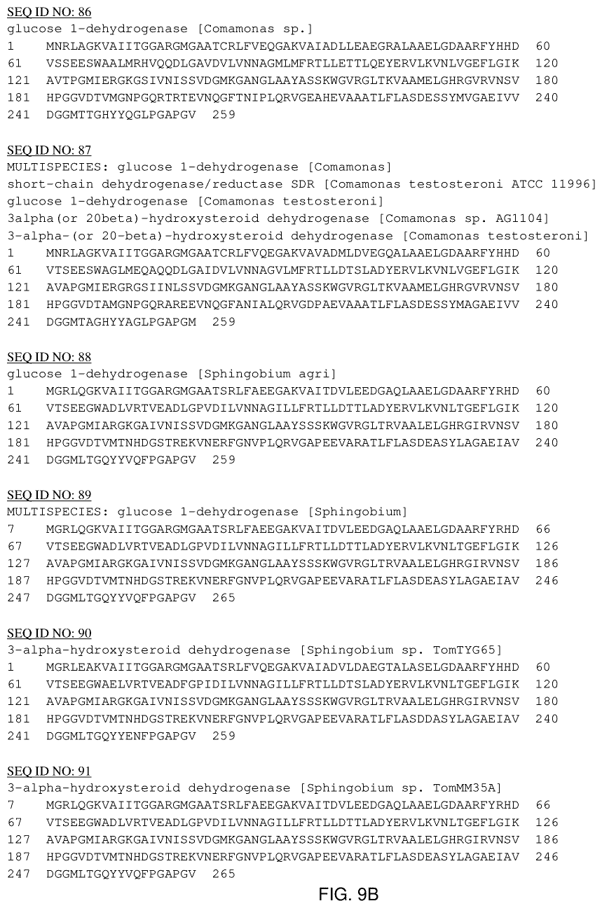

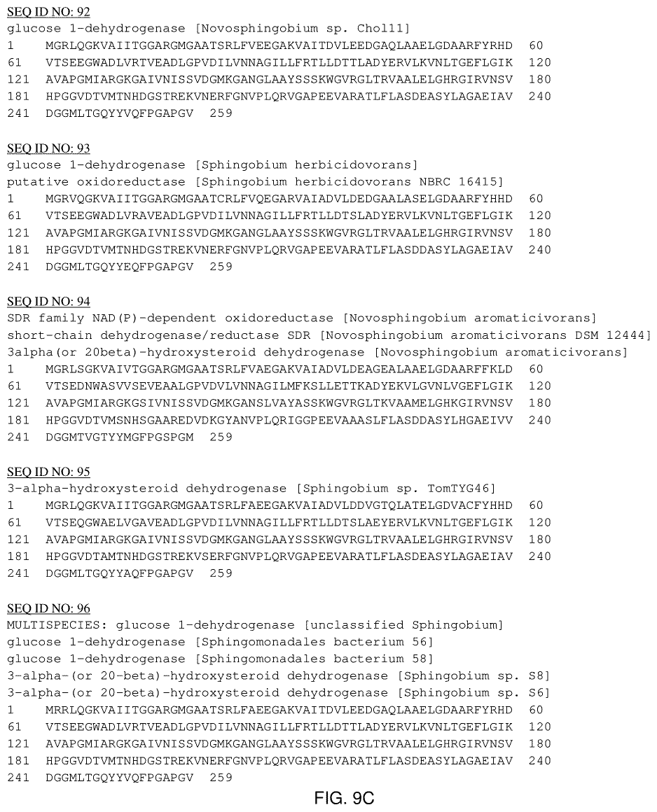

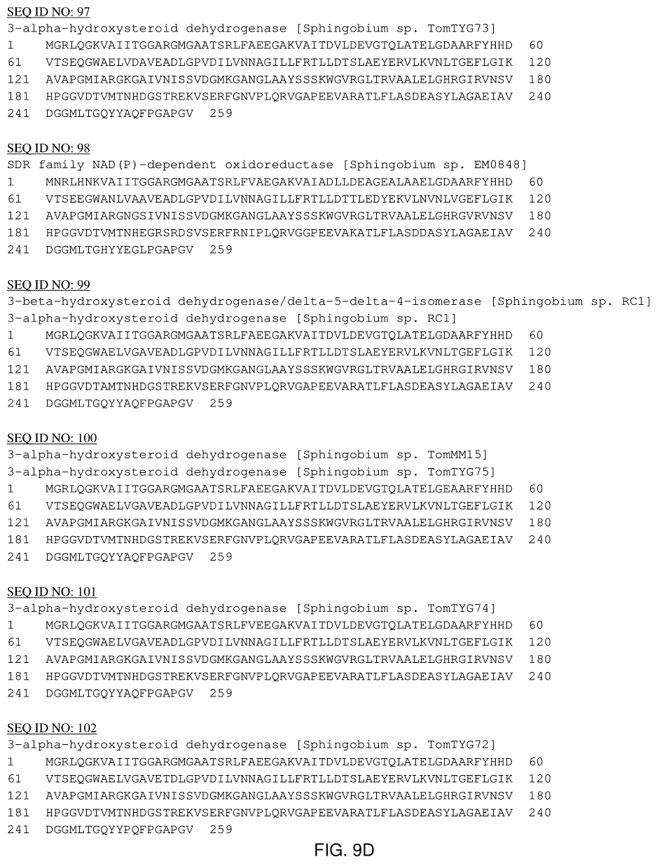

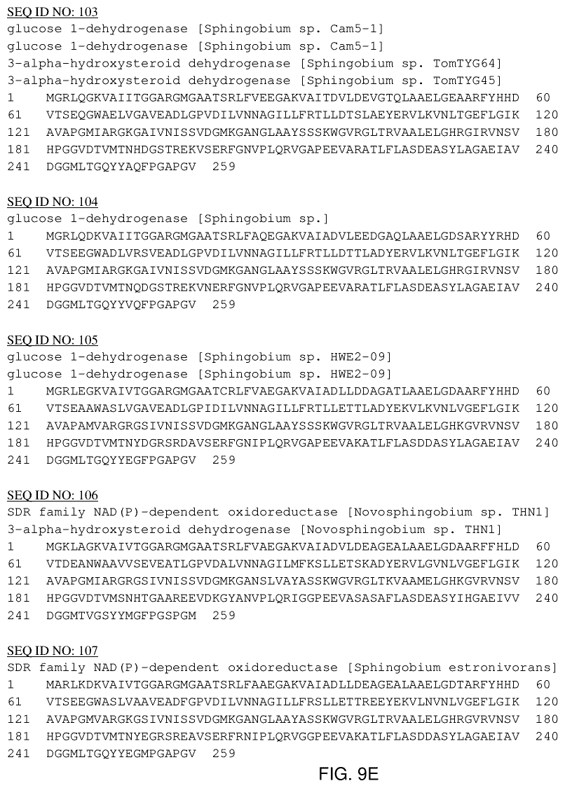

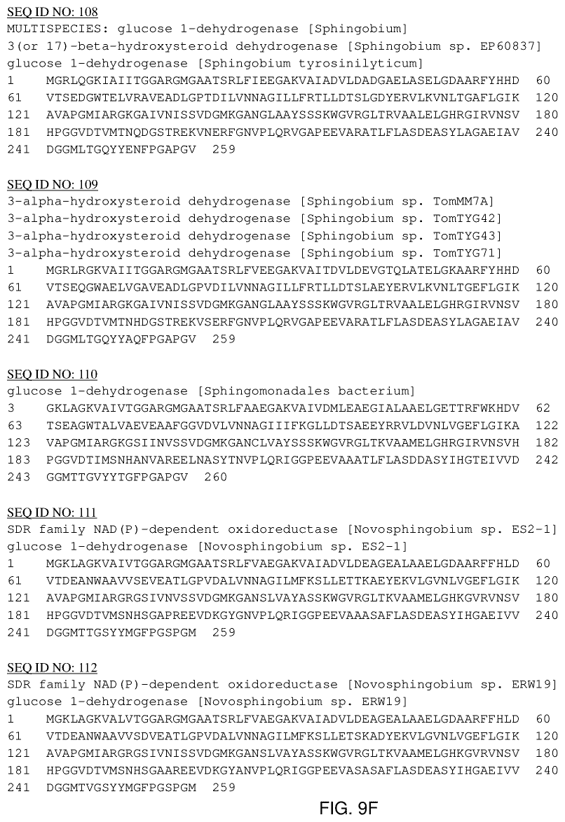

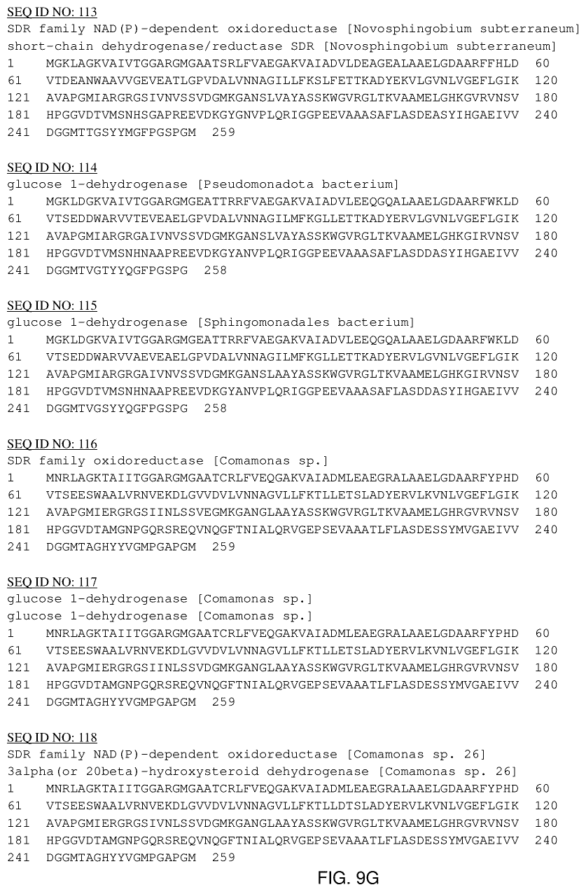

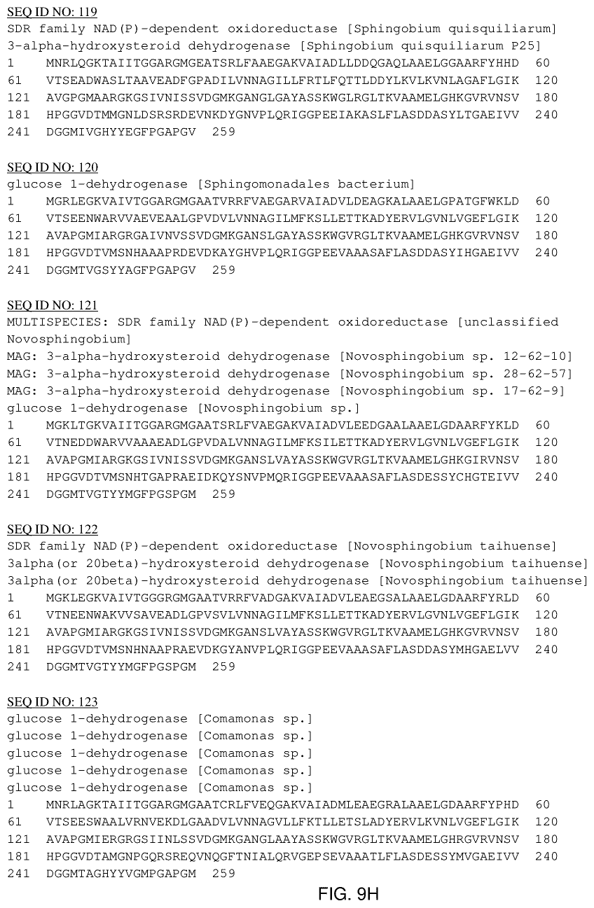

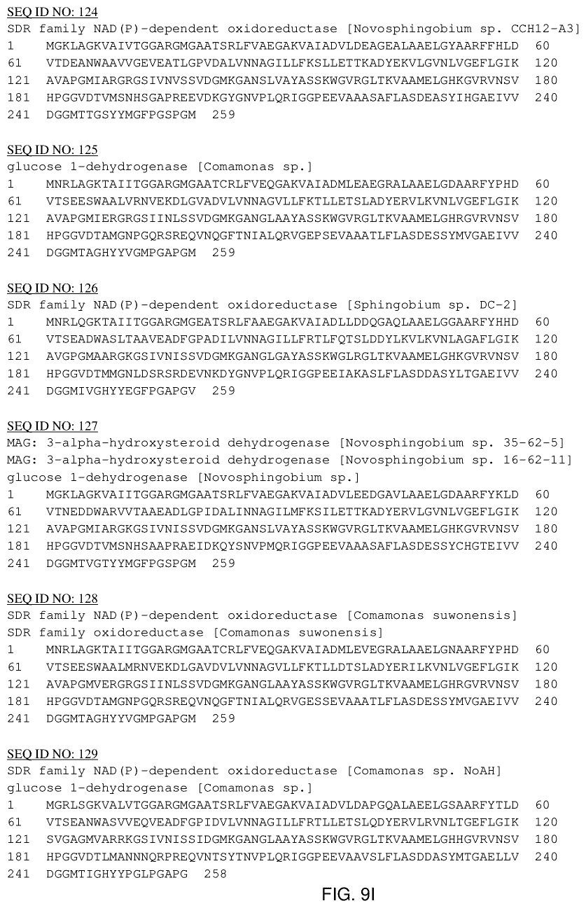

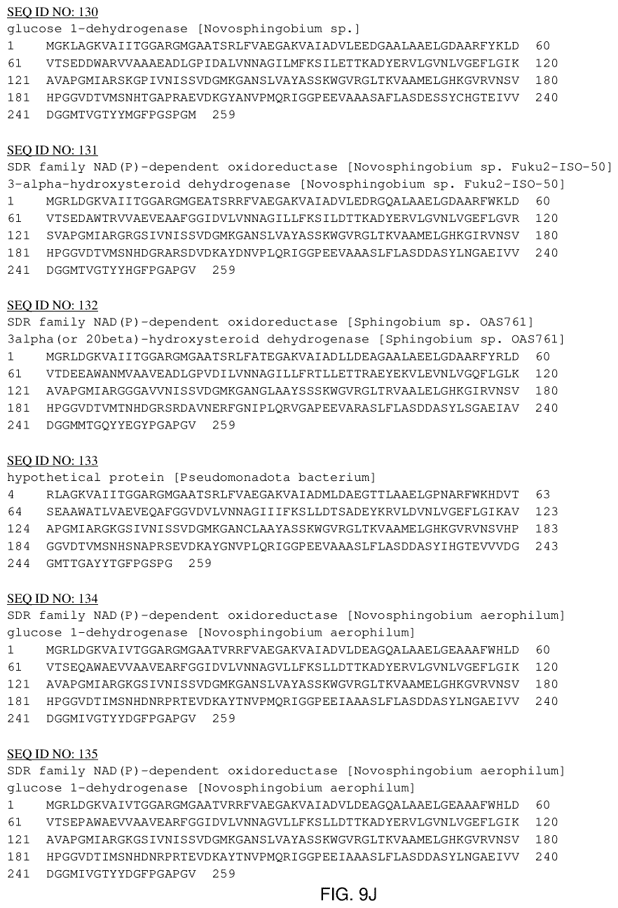

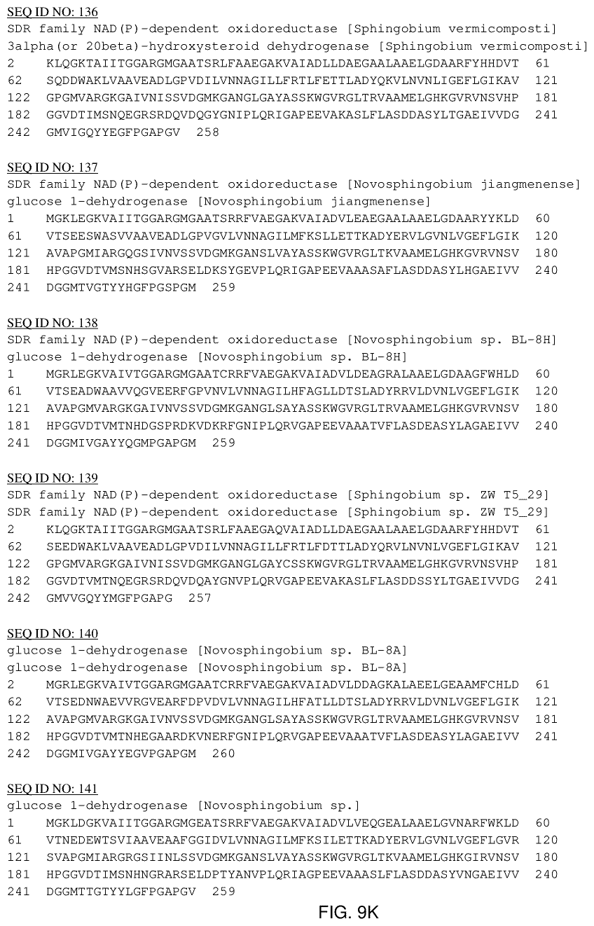

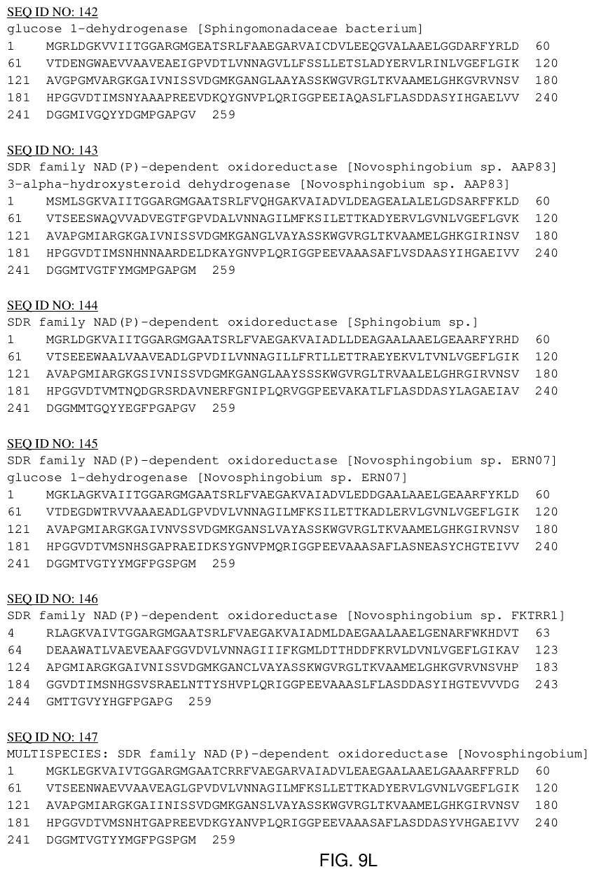

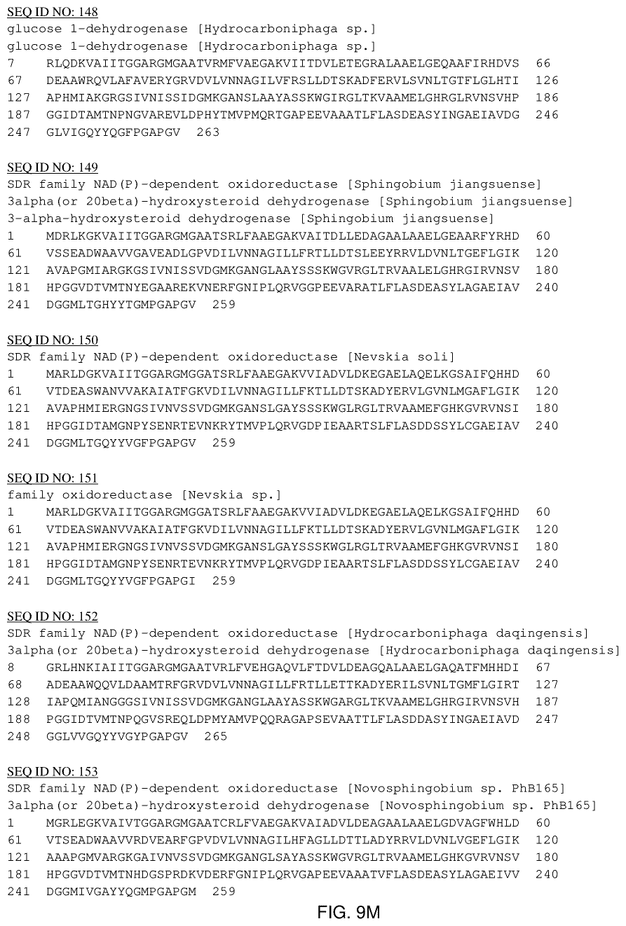

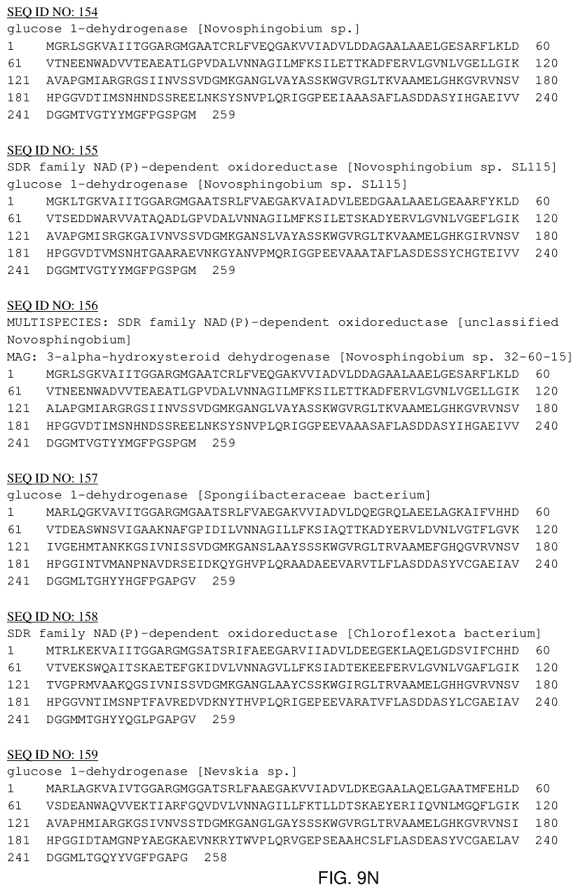

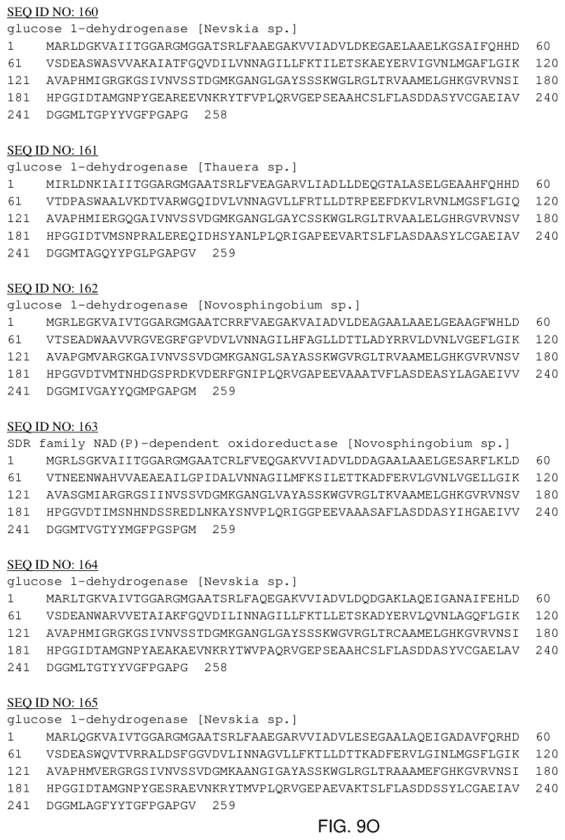

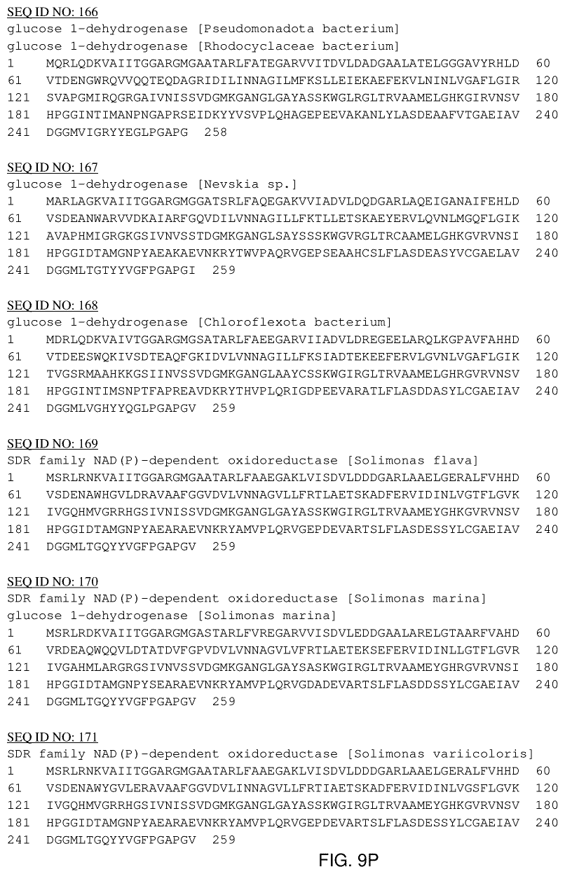

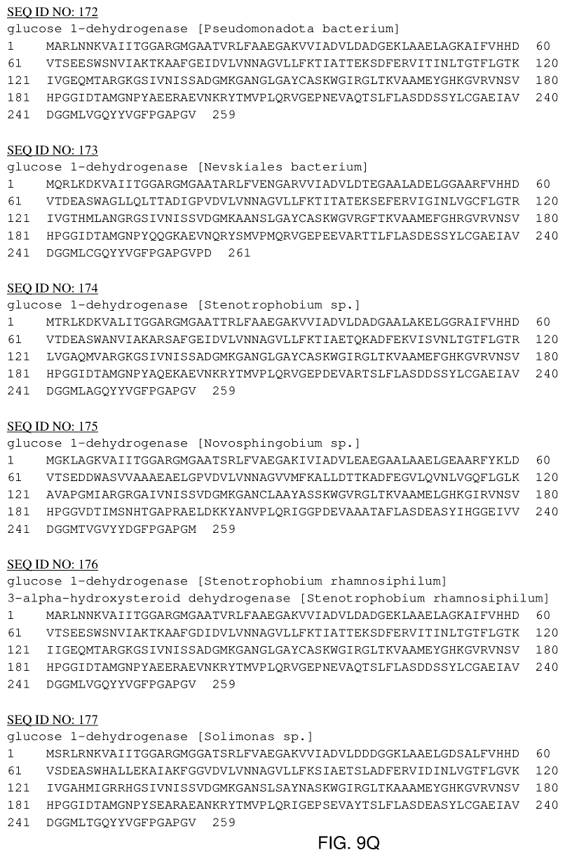

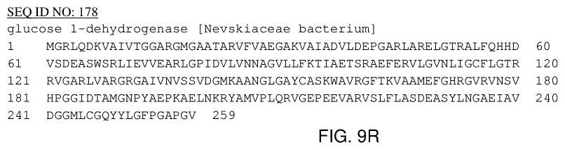

A variety of enzymes, such as dehydrogenases, can be used in various aspects. The dehydrogenase may be an enzyme able to oxidize a substrate by reducing an electron acceptor or a cofactor, usually NAD⁺/NADP⁺, or vice versa. For example, in some cases, the dehydrogenase is a 17-beta hydroxysteroid dehydrogenase. Such hydroxysteroid dehydrogenases may be able to catalyze the reduction of 17-ketosteroids and the dehydrogenation of 17-beta hydroxysteroids in steroidogenesis and steroid metabolism. A variety of organisms produce 17-beta hydroxysteroid dehydrogenases, and non-limiting examples of some organisms, and associated enzymes that they produce, are provided in FIGS. 8 and 9.

As mentioned, it has been found that certain 17-beta hydroxysteroid dehydrogenases having specific active sites able to recognize estradiol can be surprisingly effective at oxidizing estradiol to estrone. Other 17-beta hydroxysteroid dehydrogenases may not be able to specifically recognize estradiol, may not be able to oxidize estradiol to produce estrone, or may be indiscriminate in their activity. One example of such a 17-beta hydroxysteroid dehydrogenases having an active site able to recognize estradiol is the 17-beta hydroxysteroid dehydrogenase arising from Caenibius tardaugens, identified herein as EGO55_02230, which encodes an SDR family NAD (P)-dependent oxidoreductase, having the nucleotide sequence shown in SEQ ID NO: 70 or the amino acid sequence shown in SEQ ID NO: 70. In some cases, as discussed herein, such an enzyme can be used to oxidize estradiol to produce estrone and electrons, which can be determined, for example, as a current or a voltage, to determine estradiol, e.g., in solution. The enzyme may also be in solution, or immobilized on an electrode surface, etc., such as is described herein. As discussed in more detail herein, an enzyme such as a 17-beta hydroxysteroid dehydrogenase may, in some embodiments, be immobilized to the electrode.

In addition, relatively minor changes can be made to SEQ ID NO: 70 in other embodiments. For example, enzymes having structures that are at least 75%, at least 80%, at least 85%, at least 90%, at least 95%, at least 97%, at least 98%, at least 99%, etc. homologous to SEQ ID NO: 1 may be used in other cases. In addition, enzymes with no more than 10, 9, 8, 7, 6, 5, 4, 3, 2, or 1 change from SEQ ID NO: 70 can be used in certain embodiments.

In addition, in still other embodiments, other enzymes may be used, in addition to the 17-beta hydroxysteroid dehydrogenase arising from Caenibius tardaugens, or variants thereof (e.g., as discussed herein). For example, in some embodiments, a 17-beta hydroxysteroid dehydrogenase or other enzymes arising from one of the organisms shown in FIGS. 8 and 9 can be used. Such enzymes can be used in the wild-type state, or in some cases, such enzymes can be modified. For example, relatively minor changes to such enzymes may be used in certain embodiments, e.g., enzymes having structures that are at least 75%, at least 80%, at least 85%, at least 90%, at least 95%, at least 97%, at least 98%, at least 99%, etc. homologous to the wild-type sequences. In addition, enzymes with no more than 10, 9, 8, 7, 6, 5, 4, 3, 2, or 1 change from the wild-type sequences can be used in certain embodiments. In some cases, an enzyme such as any of these, or other enzymes, may be modified to have an active site able to recognize estradiol, e.g., as discussed herein.

Accordingly, in some embodiments, enzymes such as any of those described herein may have an active site that is able to recognize estradiol, or is modified such that the active site is be able to recognize estradiol. These include, but are not limited to, 17-beta hydroxysteroid dehydrogenase arising from Caenibius tardaugens, or any of the organisms and enzymes shown in FIGS. 8 and 9. In some cases, the binding of the active site of the enzyme to estradiol may be specific. For example, the specific binding may be such that a substrate binds to the enzyme more tightly than other potential substrates of the enzyme. Once bound, the enzyme may also be able to oxidize or otherwise react with estradiol. For example, in some cases, the estradiol may be oxidized to produce estrone and electrons, which can be determined, e.g., using an electrode. In some embodiments, the active site within the enzyme may be defined by certain locations within the primary structure of the enzyme which, when properly folded, creates an active site that is able to bind to certain estrogens, such as estradiol, and/or other biomolecules.

The active site can be determined using a variety of techniques. In certain embodiments, the active site can be determined by determining certain locations within the primary structure of the enzyme, e.g., as described herein. The active site may also be determined, in some cases, computationally. For example, in one set of embodiments, computational docking can be used to calculate RMSD (root mean squared distance) of a ligand (e.g., estradiol) to the amino acids in the enzyme catalytic core. Such programs are readily available commercially. For instance, in certain cases, the amino acids at position 155 (Y) and position 159 (K) of a dehydrogenase, such as a 17-beta hydroxysteroid dehydrogenase, may be useful for estradiol binding. In some embodiments, the active site may be defined by a first sequence GXXXGXG (SEQ ID NO: 2) starting within +/−10, +/−7, +/−5, +/−4, +/−3, +/−2, or +/−1 amino acids from position 13 of the dehydrogenase, and/or a second sequence LVNNAG (SEQ ID NO: 3) starting within +/−10, +/−7, +/−5, +/−4, +/−3, +/−2, or +/−1 amino acids from position 84, and/or a third sequence YXXXK (SEQ ID NO: 4) starting within +/−10, +/−7, +/−5, +/−4, +/−3, +/−2, or +/−1 amino acids from position 155.

In some cases, the first sequence may be any one of GGARGMG (SEQ ID NO: 5), GGXXGXG (SEQ ID NO: 6), GAXXGXG (SEQ ID NO: 7), GXGXGXG (SEQ ID NO: 8), GXAXGXG (SEQ ID NO: 9), GXXRGXG (SEQ ID NO: 10), GXXSGXG (SEQ ID NO: 11), GXXXGMG (SEQ ID NO: 12), or GXXXGIG (SEQ ID NO: 13). Other non-limiting examples include GGAXGXG (SEQ ID NO: 21), GGXRGXG (SEQ ID NO: 22), GGXXGMG (SEQ ID NO: 23), GXARGXG (SEQ ID NO: 24), GXAXGMG (SEQ ID NO: 25), GXXRGMG (SEQ ID NO: 26), GXARGMG (SEQ ID NO: 27), GGXRGMG (SEQ ID NO: 28), GGAXGMG (SEQ ID NO: 29), GGARGXG (SEQ ID NO: 30), GGGSGIG (SEQ ID NO: 31), GGARGIG (SEQ ID NO: 32), GGASGVG (SEQ ID NO: 33), GAGSGMG (SEQ ID NO: 34), GGSRGIG (SEQ ID NO: 35), GGGQGLG (SEQ ID NO: 36), GAGGGIG (SEQ ID NO: 37), GAAGGIG (SEQ ID NO: 38), or GGARGMG (SEQ ID NO: 39).

In some cases, the second sequence may be any one of LVNNAG (SEQ ID NO: 3), XVNNAG (SEQ ID NO: 64), LXNNAG (SEQ ID NO: 65), LVXNAG (SEQ ID NO: 66), LVNXAG (SEQ ID NO: 67), LVNNXG (SEQ ID NO: 68), or LVNNAX (SEQ ID NO: 69).

In some cases, the third sequence may be any one of YVSSK (SEQ ID NO: 14), YAXXK (SEQ ID NO: 15), YSXXK (SEQ ID NO: 16), YXAXK (SEQ ID NO: 17), YXSXK (SEQ ID NO: 18), YXXSK (SEQ ID NO: 19), or YXXAK (SEQ ID NO: 20). Other non-limiting examples include YXXSK (SEQ ID NO: 40), YVXXK (SEQ ID NO: 41), YXSXK (SEQ ID NO: 42), YXSSK (SEQ ID NO: 43), YVXSK (SEQ ID NO: 44), YVSXK (SEQ ID NO: 45), YAASK (SEQ ID NO: 46), YCASK (SEQ ID NO: 47), YSASK (SEQ ID NO: 48), YGASK (SEQ ID NO: 49), YAMSK (SEQ ID NO: 50), YSAAK (SEQ ID NO: 51), YAAAK (SEQ ID NO: 52), YSAAK (SEQ ID NO: 53), or YASSK (SEQ ID NO: 54).

In some cases, the dehydrogenase may be one of EGO55_02230 (SEQ ID NO: 1 or SEQ ID NO: 70), K8F61_00480 (SEQ ID NO: 55 or SEQ ID NO: 71), KC8_14445 (SEQ ID NO: 56 or SEQ ID NO: 72), CTATCC11996_RS21745 (SEQ ID NO: 57 or SEQ ID NO: 73), OEY_RS0121985 (SEQ ID NO: 58 or SEQ ID NO: 74), A210_RS19970 (SEQ ID NO: 59 or SEQ ID NO: 75), A9C11_RS06000 (SEQ ID NO: 60 or SEQ ID NO:76), QYC26_00545 (SEQ ID NO: 61 or SEQ ID NO: 77), EGO55_RS13560 (SEQ ID NO: 62 or SEQ ID NO: 78), IM701_17900 (SEQ ID NO: 63 or SEQ ID NO: 79), KC8_09390 (SEQ ID NO: 179 or SEQ ID NO: 180), or KC8_16655 (SEQ ID NO: 181 or SEQ ID NO: 182). In addition, in some cases, the dehydrogenase may be one of those arising from the organisms shown in FIG. 8, or any of the enzymes shown in FIG. 9.

It should be understood that each “X” in any of the sequences described herein may each independently be any suitable amino acid, and different X's within a particular given sequence may independently be the same or different. X may be, for example, alanine (“Ala” or “A”), arginine (“Arg” or “R”), asparagine (“Asn” or “N”), aspartic acid (“Asp” or “D”), cysteine (“Cys” or “C”), glutamine (“Gln” or “Q”), glutamic acid (“Glu” or “E”), glycine (“Gly” or “G”), histidine (“His” or “H”), isoleucine (“Ile” or “I”), leucine (“Leu” or “L”), lysine (“Lys” or “K”), methionine (“Met” or “M”), phenylalaine (“Phe” or “F”), proline (“Pro” or “P”), serine (“Ser” or “S”), threonine (“Thr” or “T”), tryptophan (“Trp” or “W”), tyrosine (“Tyr” or “Y”), or valine (“Val” or “V”). In some cases, other amino acids may be present.

In some embodiments, one or more of the sequences may be annotated. Possible annotations of these include, but are not limited to, 17-beta-estradiol 17-dehydrogenase, 3-ketosteroid-9-alpha-hydroxylase oxygenase subunit, 3-ketosteroid-9-alpha-hydroxylase reductase subunit, 3-oxo-5-alpha-steroid 4-dehydrogenase, short-chain dehydrogenase/reductase, 3-alpha-hydroxysteroid dehydrogenase, 3-ketosteroid-delta-1-dehydrogenase, acetaldehyde dehydrogenase, cholesterol oxidase, 3-ketosteroid-9alpha-hydroxylase, dioxygenase, 3,4-DHSA dioxygenase, monooxygenase, acetyl-coA acetyltransferase, 3-ketoacyl-CoA thiolase, 17-beta-hydroxysteroid dehydrogenasse, short-chain dehydrogenase/reductase, flavin-dependent monooxygenase, 17-beta-hydroxysteroid dehydrogenase, estrone 4-hydroxylase, 4-hydroxyestrone 4,5-dioxygenase, short-chain dehydrogenase/reductase, hydroxysteroid dehydrgoenase, 3-ketosteroid-delta-dehydrogenase, rieske dioxygenase, catechol 2,3-dioxygenase, 2,3-dihydroxyphenylproprionate, 2,3-dihydroxicinnsmic acid 1,2-dioxygenase, biphenyl-2,3-diol 1,2-dioxygenase, meta-cleavage enzyme, SDR family NAD (P)-dependent oxidoreductase, SDR family oxidoreductase, or 3-oxoacyl-ACP reductase family protein.

In addition, it should be understood that in other embodiments, other enzymes may be used instead of dehydrogenases such as 17-beta hydroxysteroid dehydrogenases. For example, the enzyme may be an oxidase, or an oxidoreductase, such as a class EC1 oxidoreductase. In some embodiments, an enzyme may be immobilized relative to a surface, e.g., the surface of an electrode. A variety of systems may be used to immobilize the enzyme to the surface. For example, in one embodiment, the enzyme is immobilized on the electrode with chitosan. As another example, the enzyme may be immobilized on an electrode by crosslinking a redox polymer. For example, an aqueous mixture containing enzyme, redox polymer, and a crosslinking agent may be applied onto an electrode and dried to form a film or coating on the electrode. As yet another example, an enzyme may be immobilized on an electrode using a self-assembled monolayer (SAM), e.g., a SAM comprising chemisorbed alkanethiols. In some cases, the electrode may comprise gold, and the SAM may be chemisorbed using groups such as thiols, sulfates, sulfonates, phosphates, selenides, etc. In another example, an enzyme may be immobilized onto an electrode using glutaraldehyde. In addition, in some cases, an enzyme maybe deposited on an electrode without an immobilizing agent.

In some embodiments, the electrode is metallic. Non-limiting examples include gold, silver, platinum, titanium, copper, or palladium. In some embodiments, the electrode is non-metallic. In some embodiments, the non-metallic electrode comprises carbon (e.g., glassy carbon, carbon screen printed conductive inks, graphite, carbon cloth, edge-plane pyrolytic graphite, etc.). Other examples include silver/silver chloride, poly(3,4-ethylenedioxythiophene) polystyrene sulfonate or PEDOT:PSS. In some cases, the electrode has a medical wire electrode configuration, for example, for certain on-body applications. The device may have one, two, or more electrodes. For example, the device may include a working electrode, a reference electrode, a counter electrode, etc. Those of ordinary skill in the art will be aware of such electrodes, if present, and their function. A working electrode can be, for example, carbon, glassy carbon, metal, metal oxides, a mixture of carbon and metal or metal oxides, or the like. In one example, the working electrode is a glassy carbon electrode. A reference electrode can be, for example, a saturated calomel reference electrode (SCE), Ag/AgCl, Ag/Ag, or saturated Hg₂Cl₂, etc. In some embodiments, the counter or reference electrode can be, for example, a metal such as gold, silver, platinum, titanium, or stainless steel, such as a metal wire counter or reference electrode. In some embodiments, the working and/or the reference electrodes, and optionally the counter electrode, are screen printed electrodes (SPE), interdigitated electrodes (IDEs), and/or organic electrochemical transistors (OECTs), etc.

The electrodes, such as an active electrode, can be formed, for example, by coating a metal with a formulation, and drying the coating on the metal. For example, a fine platinum wire can be coated with a formulation, and the coating dried in place. The coated wire can be arranged in a syringe or other suitable flow cell or channel device that can be placed, for example, in-line or into the flow of an analyte stream to be monitored for hormone concentration. The formulation may include an enzyme, e.g., as discussed herein. In some cases, the formulation may include other components such as any of those discussed herein; for example, electron carriers, redox mediators, components for immobilizing an enzyme relative to a surface, etc.

In some embodiments, various electrodes, e.g., comprising platinum, silver, carbon, gold, titanium, and Ag/AgCl ink, etc. can be used in screen-printing methods, and/or photolithographically patterned metal vapor deposition methods, e.g., to form such electrodes. These electrodes can be used, for example, in electrode strips, biochips, or other configurations. The biosensor can be, for example, a screen-printed (e.g., a screen-printed electrode (or SPE)) or photolithographically patterned three-electrode transducer, e.g., with a carbon or platinum working electrode. In some embodiments, the biosensor can be screen-printed or ink-jet printed. Other transducer configurations also can be used on other embodiments.

In certain aspects, the transfer of electrons between an electrode and an enzyme, e.g., such as discussed herein, may result in the flow of electrons and current generation, which may be correlated with the response of the enzyme to a substrate, such as estrogen. Such electrons may be transferred directly, e.g., by direct electron transfer to an electrode, and/or indirectly, e.g., electrons may be transferred via one or more electron carriers or cofactors, such as oxygen (O₂), redox mediators or the like. As discussed, in certain embodiments, the electrons determined by the electrode may correspond to the amount of the estrogen or other biomolecule present.

For example, in some embodiments, an electron carrier may be used to facilitate the transfer of electrons to or from an electrode. The electron carrier may be in solution, immobilized relative to the electrode, immobilized relative to an enzyme (e.g., via a covalent bond), or the like. Electron carriers may include redox mediators, charge transfer enhancing media, or other carriers such as any of those described herein. In certain embodiments where an electron carrier is immobilized relative to an electrode, any suitable immobilization technique may be used, e.g., including any of those that can be used for immobilizing an enzyme relative to a surface discussed herein, for example, chitosan, redox polymers, SAM, glutaraldehyde, etc. In various embodiments, the technique for immobilizing the electron carrier and the enzyme may independently be the same or different.

In one set of embodiments, the electrons may be transferred using a redox mediator, such as an electronically active mediator. In some cases, a redox reaction catalyzed by an enzyme may transfer electrons to or from a redox mediator, either directly or indirectly. The redox mediator may transfer electrons to or from an electrode, e.g., to produce a determinable flow of electrons, e.g., which can be determined as a current. In some embodiments, redox mediators are electroreducible and/or electrooxidizable ions or molecules. In some embodiments, a redox reaction may result in the transfer of an electron from a reduced form of a redox mediator to hydrogen peroxide, resulting in its oxidation. In some embodiments, the redox mediator is reduced at the electrode, producing an electron that can be determined at the electrode.

Non-limiting examples of redox mediators include cytochromes, quinones, aminophenols, electron-acceptor aromatic compounds (e.g., TTF or tetratiafulvalene, NMP or N-methylphenazine, etc.), electron-donor aromatic compounds (e.g., TCNQ or tetrakyano-p-quinodimethane), organic conductive salts (e.g., TTF.TCNQ or tetratiafulvalene-7,7,8,8-tetrakyano-p-quinodimethane and NMP.TCNQ or N-methylphenylene-7,7,8,8-tetracyano-p-quinodimethane), organic dyes, metallocenes, organometallic complexes of osmium, ruthenium, inorganic iron complexes, etc. Additional examples of redox mediators include ferricyanide ferrocene, 1,1-dimethyl-ferrocene, hexacyanoferrate or hexacyanoferrate, or platinum. In some embodiments, the redox mediator is Amplex® UltraRed (AUR). Still additional non-limiting examples include cytochrome A3, cytochrome C3, cytochrome b, ubiquitone, vitamin K2, rubredoxin, flavoproteins, FAD-FADH₂, FMN-FNH₂, NAD+-NADH, NADP+-NADPH, PQQ-PQQH₂, ferricyanide (hexacyanoferrate III), 2,6-dichlorophenol, indophenol, ferrocene, phenazine, methosulphate, methylene blue, phtalocyannine, phenosafranine, benzyl violet, methyl violet, ferredoxin, Prussian blue, Nile blue, Meldola's Blue, NQSA, potassium hexacyanoferrate, potassium ferricyanide, potassium ferrocyanide, PMS, dichlorophenolindophenol, p-benzoquinone, o-phenylenediamine, 3,4-dihydroxybenzaldehyde, potassium hexacyanoferrate (II), tetracyanoquinodimethane, cobalt (II) phtalocyanine, or platinum, etc. Further examples include benzoquinone, anthraquinone, phenazine methosulfate, phenazine ethosulfate, methylene blue, thionine, ferrocene methanol, 1,1′-dimethylferrocene, ferrocenecarboxylic acid, methyl viologen, benzyl viologen, potassium hexacyanoferrate, ferrocene, cobaltocene, ruthenium hexamine, osmium complexes, nicotinamide adenine dinucleotide, nicotinamide adenine dinucleotide phosphate, flavin adenine dinucleotide, flavin mononucleotide, cytochrome c, pyrroloquinoline quinone, iron-sulfur clusters, hemes, blue copper (cysteine-Cu) proteins, polyaniline, polypyrrole, polythiophene, polythiophene derivatives, poly(benzimidazobenzophenanthroline), poly(p-phenylene vinylene). Additional non-limiting examples of redox mediators include various ferricyanide compounds. For example, in some embodiments, the redox mediator comprises a ferricyanide compound which is reducible in the presence of an electron from hydrogen peroxide to produce a ferrocyanide compound. In some embodiments, the electronically active mediator comprises iron(II,III) hexacyanoferrate(II,III) (e.g., Prussian blue).

In addition, in some embodiments, the electron carrier may include a charge transfer enhancing medium. The charge transfer enhancing medium may allow direct transfer of electrons directly through the medium. Examples of such media include, but are not limited to, carbon materials (e.g., graphene, carbon nanotubes (single or multi-walled), etc.), nanoparticles, conductive polymers, etc. Other non-limiting examples include alkanethiols, graphene oxides, non-conductive polymers, hydrogels, nafion, aquion, conductive polymers, poly(3,4-ethylenedioxythiophene):polystyrene sulfonate (PEDOT:PSS), poly(benzimidazobenzophenanthroline):poly(ethyleneimine) (BBL:PEI), or the like. The charge transfer enhancing medium may be immobilized to the surface of an electrode in certain embodiments. In some cases, an enzyme and/or a mediator may be immobilized to the charge transfer enhancing medium.

In some cases, at least one further component may be present, such as an intermediate redox enzyme. In some embodiments, the further component may be: one or more additional enzymes forming an enzymatic pathway utilizing the product or by-product of the initial redox-enzyme reaction to thereby generate a photometrically or electrochemically detectable product or by-product; at least one signal mediator, etc. The signal mediators may include indicators, such as a pH-change indicators; electron transfer mediators; photometric mediators, and other components.

In some embodiments, an electrochemically detectable cofactor may be used, such as NADH or FAD, or a by-product may be generated, such as H₂O₂ or electrons, e.g., during the enzymatic reaction with hormone. Other non-limiting examples of cofactors include PQQ, NAD⁺, NADP⁺, NADH, NADPH, FAD, FMN, cytochrome c, etc.

For illustrative purposes only, an enzyme can be conjugated to an electroactive molecule and the analyte probe attached or on the surface of a conducting surface of a semiconductor device, such that when the enzyme is bound to hormone, the electroconductive molecule conjugated to the enzyme and hormone are in close proximity to allow electron transfer, and the flow of electrons to the electrode can be determined.

U.S. Pat. No. 11,801,000 is incorporated herein by reference in its entirety. In addition, U.S. Pat. Apl. Ser. No. 63/735,965, filed Dec. 19, 2024, is also incorporated herein by reference in its entirety.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A device, comprising:
a 17-beta hydroxysteroid dehydrogenase, configured to specifically react estradiol to produce estrone and electrons, in electrical communication with an electrode.

2. The device of claim 1, wherein the dehydrogenase is immobilized relative to the electrode.

3. The device of claim 1, wherein the dehydrogenase is in solution.

4-6. (canceled)

7. The device of claim 1, wherein the electrode is in contact with a solution containing estradiol.

8. The device of claim 7, wherein the solution comprises a fluid arising from a subject.

9. The device of claim 8, wherein the subject is human.

10-30. (canceled)

31. The device of claim 1, wherein the 17-beta hydroxysteroid dehydrogenase arises from Caenibius tardaugens.

32. The device of claim 1, wherein the 17-beta hydroxysteroid dehydrogenase is EGO55_02230 (SEQ ID NO: 70).

33. The device of claim 1, wherein the 17-beta hydroxysteroid dehydrogenase is K8F61_00480 (SEQ ID NO: 71), KC8_14445 (SEQ ID NO: 72), CTATCC11996_RS21745 (SEQ ID NO: 73), OEY_RS0121985 (SEQ ID NO: 74), A210_RS19970 (SEQ ID NO: 75), A9C11_RS06000 (SEQ ID NO: 76), QYC26_00545 (SEQ ID NO: 77), EGO55_RS13560 (SEQ ID NO: 78), IM701_17900 (SEQ ID NO: 79), KC8_09390 (SEQ ID NO: 180), or KC8_16655 (SEQ ID NO: 182).

34. The device of claim 1, wherein the 17-beta hydroxysteroid dehydrogenase is an enzyme listed in FIG. 8.

35. The device of claim 1, wherein the 17-beta hydroxysteroid dehydrogenase is any of SEQ ID NOs. 86-178.

36. The device of claim 1, further comprising an electron carrier in electronic communication with the 17-beta hydroxysteroid dehydrogenase.

37-59. (canceled)

60. The device of claim 1, wherein the device is configured to determine the reaction using chronoamperometry, intermittent pulse amperometry, double potential-step amperometry, chronopotentiometry, transient potentiometry, cyclic voltammetry, differential pulse voltammetry, square wave voltammetry, alternating-current voltammetry, fast scan voltammetry, staircase voltammetry, electrochemical impedance spectroscopy, or electrochemical impedance spectroscopy.

61. The device of claim 1, wherein the device is a continuous monitoring device.

62-70. (canceled)

71. A method, comprising:
oxidizing estradiol to produce estrone and electrons using a 17-beta hydroxysteroid dehydrogenase;
transferring the electrons to an electrode; and
determining the estradiol based on the transfer of electrons to the electrode.

72. The method of claim 71, wherein the 17-beta hydroxysteroid dehydrogenase has at least 80% homology to EGO55_02230 (SEQ ID NO: 70).

73. The method of claim 71, wherein the 17-beta hydroxysteroid dehydrogenase has an active site comprising:
a first sequence GXXXGXG (SEQ ID NO: 2) starting within +/−5 amino acids from position 13,
a second sequence LVNNAG (SEQ ID NO: 3) starting within +/−5 amino acids from position 84, and
a third sequence YXXXK (SEQ ID NO: 4) starting within +/−5 amino acids from position 155.

74. The method of claim 71, wherein transferring the electrons to an electrode comprises directly transferring the electrons from the 17-beta hydroxysteroid dehydrogenase to the electrode.

75. The method of claim 71, wherein transferring the electrons to an electrode comprises:
transferring electrons from the 17-beta hydroxysteroid dehydrogenase to an electron carrier; and
transferring electrons from the electron carrier to the electrode.

76. The method of claim 75, wherein the electron carrier comprises a mediator.

77-86. (canceled)

87. A device, comprising:
an enzyme in electrical communication with an electrode, wherein the enzyme has at least 80% homology to EGO55_02230 (SEQ ID NO: 70), and an active site comprising:
a first sequence GXXXGXG (SEQ ID NO: 2) starting within +/−5 amino acids from position 13,
a second sequence LVNNAG (SEQ ID NO: 3) starting within +/−5 amino acids from position 84, and
a third sequence YXXXK (SEQ ID NO: 4) starting within +/−5 amino acids from position 155.

88. The device of claim 87, wherein the dehydrogenase is immobilized relative to the electrode.

89. The device of claim 87, wherein the dehydrogenase is in solution.

90-94. (canceled)
